SEQUENCE LISTING (11 sequences)

SEQ ID NO: 1 - DNA, 180 bp, Homo Sapiens
cagcggtcttagggaatttccccccgcgatgtcccggcgcgccagttcgctgcgcacacttcgctgcggtcctcttcctgctgtctgtttactccctaggccccgctggggacctgggaaagagggaaaggcttccccggccagctgcgcggcgactccggggactccagggcgcccctc

SEQ ID NO: 2 - DNA, 13 bp, Homo Sapiens
gaaacatcgcgaa

SEQ ID NO: 3 - DNA, 15 bp, Homo Sapiens
gaaacatcgcgaaaa

SEQ ID NO: 4 - DNA, 16 bp, Homo Sapiens
atcgccgcgcaactaa

SEQ ID NO: 5 - DNA, 15 bp, Homo Sapiens
aaaacatcacaaaaa

SEQ ID NO: 6 - DNA, 16 bp, Homo Sapiens
atcaccacacaactaa

SEQ ID NO: 7 - DNA, 15 bp, Homo Sapiens
ctaacgcgccgaaac

SEQ ID NO: 8 - DNA, 14 bp, Homo Sapiens
ccactacaatccca

SEQ ID NO: 9 - DNA, 12 bp, Homo Sapiens
caccacacaact

SEQ ID NO: 10 - DNA, 14 bp, Homo Sapiens
gcaactaagcaacg

SEQ ID NO: 11 - DNA, 14 bp, Homo Sapiens
gcaacgaactaacg

(19) United States
(12) Patent Application Publication
(10) Pub. No.: US 2004/0086944 A1
(43) Pub. Date: May 6, 2004

(54) Detection of methylated dna molecules

(76) Inventors: Geoffrey Walter Grigg, New South Wales (AU); Peter Molloy, New South Wales (AU); Douglas Spencer Millar, New South Wales (AU)

Correspondence Address: CAMPBELL & FLORES LLP, 4370 LA JOLLA VILLAGE DRIVE, 7TH FLOOR, SAN DIEGO, CA, 92122, US

(21) Appl. No.: 10/416,637

(22) PCT Filed: Nov. 12, 2001
(86) PCT No.: PCT/AU01/01465

(30) Foreign Application Priority Data
Nov. 13, 2000 (AU) PR 1425

(51) Int. Cl. G01N 33/53

(52) U.S. Cl. 435/7.1

(57) ABSTRACT

A method for detecting presence of a target DNA in a sample, the method comprising: (a) treating a sample containing DNA with an agent that modifies unmethylated cytosine; (b) providing to the treated sample a detector ligand capable of binding to a target region of DNA and allowing sufficient time for a detector ligand to bind to a target DNA; and (c) measuring binding of the detector ligand to DNA in the sample to determine the presence of the target DNA in a sample.

### TECHNICAL FIELD

This invention relates to DNA hybridisation assays and in particular to an improved oligonucleotide or peptide nucleic acid (PNA) assay. The invention also relates to methods for distinguishing specific base sequences including 5-methyl cytosine bases in DNA using these assays.

### BACKGROUND ART

A number of procedures were available for the detection of specific nucleic acid molecules. These procedures typically depend on sequence-dependent hybridisation between the target DNA and nucleic acid probes which may range in length from short oligonucleotides (20 bases or less) to sequences of many kilobases.

For direct detection, the target DNA is most commonly separated on the basis of size by gel electrophoresis and transferred to a solid support prior to hybridisation with a probe complementary to the target sequence (Southern and Northern blotting). The probe may be a natural nucleic acid or analogue such as PNA or locked nucleic acid (LNA). The probe may be directly labelled (eg. with ³²P) or an indirect detection procedure may be used. Indirect procedures usually rely on incorporation into the probe of a “tag” such as biotin or digoxigenin and the probe is then detected by means such as enzyme-linked substrate conversion or chemiluminescence.

Another method for direct detection of nucleic acid that has been used widely is “sandwich” hybridisation. In this method, a capture probe is coupled to a solid support and the target DNA, in solution, is hybridised with the bound probe. Unbound target DNA is washed away and the bound DNA is detected using a second probe that hybridises to the target s quences. Detection may use direct or indirect methods as outlined above. The “branched DNA” signal detection system is an example that uses the sandwich hybridization principl (Urdea Ms Branched DNA signal amplification. Biotechnology 12: 926-928).

A rapidly growing area that us s nucleic acid hybridisation for direct detection of nucleic acid sequences is that of DNA micro-arrays (Young RA Biomedical discovery with DNA arrays. Cell 102: 9-15 (2000); Watson, A New tools. A new breed of high tech detectives. Science 289:850-854 (2000)). In this process, individual nucleic acid species, that may range from oligonucleotides to longer sequences such as cDNA clones, were fixed to a solid support in a grid pattern. A tagged or labelled nucleic acid population is then hybridised with the array and the level of hybridisation with each spot in the array is quantified. Most commonly, radioactively or fluorescently-labelled nucleic acids (eg. cDNAs) were used for hybridisation, though other detection systems were employed.

The most widely used method for amplification of specific sequences from within a population of nucleic acid sequences is that of polymerase chain reaction (PCR) (Dieffenbach C and Dveksler G eds. PCR Primer: A Laboratory Manual. Cold Spring Harbor Press, Plainview N.Y.). In this amplification method, oligonucleotides, generally 15 to 30 nucleotides in length on complementary strands and at either end of the region to be amplified, were used to prime DNA synthesis on denatured single-stranded DNA. Successive cycles of denaturation, primer hybridisation and DNA strand synthesis using thermostable DNA polymerases allows exponential amplification of the sequences between the primers. RNA sequences can be amplified by first copying using reverse transcriptase to produce a cDNA copy. Amplified DNA fragments can be detected by a variety of means including gel electrophoresis, hybridisation with labelled probes, use of tagged primers that allow subsequent identification (eg. by an enzyme linked assay), use of fluorescently-tagged primers that give rise to a signal upon hybridisation with the target DNA (eg. Beacon and TaqMan systems).

As well as PCR, a variety of other techniques have been developed for detection and amplification of specific sequences. One example is the ligase chain reaction (Barany F Genetic disease detection and DNA amplification using cloned thermostable ligase. Proc. Natl. Acad. Sci. USA 88:189-193 (1991)).

Currently the method of choice to detect methylation changes in DNA, such as w re found in the GSTP1 gene promoter in prostate cancer, were dependent on PCR amplification of such sequ nces after bisulfite modification of DNA. In bisulfite-treated DNA, cytosines were converted to uracils (and hence amplified as thymines during PCR) while methylat d cytosines were non-reactive and remain as cytosines (Frommer M, McDonald L E, Millar D S, Collis C M, Watt F, Grigg G W, Molloy P L and Paul C L. A genomic sequencing protocol which yields a positive display of 5-methyl cytosine residues in individual DNA strands. PNAS 89: 1827-1831 (1992); Clark S J, Harrison J, Paul C L and Frommer M. High sensitivity mapping of methylated cytosines. Nucleic Acids Res. 22: 2990-2997 (1994)). Thus (after bisulfite treatment) DNA containing 5-methyl cytosine bases will be different in sequence from the corresponding unmethylated DNA. This report results in the basis of the bisulfite method for sequencing 5-methyl cytosine residues in DNA. Surprisingly, several years later this assay was used as the basis of a PCR assay for the methylation status of CpG islands in U.S. Pat. No. 5,786,146. Primers may be chosen to amplify non-selectively a region of the genome of interest to determine its methylation status, or may be designed to selectively amplify sequences in which particular cytosines were methylated (Herman J G, Graff J R, Myohanen S, Nelkin B D and Baylin S B. Methylation-specific PCR: a novel PCR assay for methylation status of CpG islands. PNAS 93:9821-9826 (1996)).

Alternative methods for detection of cytosine methylation include digestion with restriction enzymes whose cutting is blocked by site-specific DNA methylation, followed by Southern blotting and hybridisation probing for the region of interest. This approach is limited to circumstances where a significant proportion (generally >10%) of the DNA is methylated at the site and where there is sufficient DNA, usually 10 μg, to allow for detection. Digestion with restriction enzymes whose cutting is blocked by site-specific DNA methylation, followed by PCR amplification using primers that flank the restriction enzyme site(s). This method can utilise smaller amounts of DNA but any lack of complete enzyme digestion for reasons other than DNA methylation can lead to false positive signals.

Recently, peptid nucleic acids (PNA) in which the entire deoxyribose-phosphate backbon has been exchanged with a structurally homomorphous uncharged polyamide backbone composed of N-(2-aminoethyl)glycine units have been developed (Ray A and Norden B. Peptide nucleic acid (PNA): its medical and biotechnical applications and for th future. FASEB J 14: 1041-1060 (2000)). These compounds contain the same number of backbone bonds between the bases (i.e. 6) and the same number of bonds from the backbone to the base (i.e. 3) as in DNA. PNA oligomers have been found to bind with high affinity and sequence specificity to both complementary RNA and DNA and a number of oligonucleotide-dependent enzymatic functions have been inhibited on forming PNA/DNA or PNA/RNA complexes. PNAs demonstrate a higher binding affinity than their equivalent oligonucleotides and mismatches of PNAs with complementary nucleotide sequences cause a more profound lowering of melting temperature than is seen with oligonucleotides. PNAs have also shown a number of special properties, one of which is that homopyrimidine PNAs bind to double-stranded DNA with displaced strand analogous to a D-loop. More recently, Neilsen (Nielsen PE. Peptide nucleic acids as therapeutic agents. Curr. Open Struct. Biol. 9: 353-357 (1999) has reported that a homopurine PNA binds to double-stranded DNA with displacement of the non-complementary strand, resulting in formation of a PNA/DNA duplex and a displaced D-loop. However, unlike homopyrimidine PNAs, the homopurine PNA/DNA duplex is not then further stabilised by triplex formation. Hence,. PNA offers both antisense and antigene strategies for regulating gene expression.

The present inventors have now developed methods utilizing ligands for the sensitive and specific detection of DNA which do not require PCR amplification.

### DISCLOSUR OF INVENTION

In a first aspect, the present invention provides a method for detecting presence of a target DNA in a sample, the method comprising:

(a) treating a sample containing DNA with an agent that modifies unmethylated cytosine;

(b) providing to the treated sample a detector ligand capable of binding to a target region of DNA and allowing sufficient time for a detector ligand to bind to a target DNA; and

(c) measuring binding of the detector ligand to DNA in the sample to determine the presence of the target DNA in a sample.

In a second aspect, the present invention provides a method for estimating extent of methylation of a target DNA in a sample, the method comprising:

(a) treating a sample containing DNA with an agent that modifies unmethylated cytosine;

(b) providing to the treated sample a detector ligand capable of distinguishing between methylated and unmethylated cytosine of DNA and allowing sufficient time for a detector ligand to bind to a target DNA; and

(c) detecting binding of the detector ligand to DNA in the sample such that the degree or amount of binding is indicative of the extent of methylation of the target DNA.

In step (b), two detector ligands can be used where one ligand is capable of binding to a region of DNA that contains one or more methylated cytosines and the other ligand capable of binding to a corresponding region of DNA that contains no methylated cytosines. As a sample can contain many copies of a target DNA, often the copies have different amounts of methylation. Accordingly, the ratio of binding of the two ligands will be proportional to the degree of methylation of that DNA target in the sample. The two ligands can be added together in th one test or can be added in separate duplicate tests. Each ligand can contain a uniqu marker which can be detected concurrently or separately in the one test or have th same marker and detected individually in separat tests.

In a third aspect, the invention provides a method for detecting the presence of a target DNA in a sample, the method comprising:

(a) treating a sample containing DNA with an agent that modifies unmethylated cytosine;

(b) providing a support to which is bound a capture ligand which is capable of recognising a first part of a target DNA sequence;

(c) contacting the support with the treated sample for sufficient time to allow DNA to bind to a capture ligand such that target DNA in the sample binds to the support via the capture ligand;

(d) contacting the support with a detector ligand capable of recognising a second part of the target DNA sequence and allowing sufficient time for a detector ligand to bind to a target DNA bound to a support; and

(e) measuring binding of the detector ligand to DNA bound to the support to determine the presence of the target DNA in the sample.

In a fourth aspect, the present invention provides a method for estimating extent of methylation of a target DNA in a sample, the method comprising:

(a) treating a sample containing DNA with an agent that modifies unmethylated cytosine;

(b) providing a support to which is bound a capture ligand which is capable of recognising a first part of a target DNA sequence;

(c) contacting the support with the treated sample for sufficient time to allow DNA to bind to a capture ligand such that target DNA in the sample binds to the support via the capture ligand;

(d) contacting the support with a detector ligand capable of distinguishing between methylated and unmethylated cytosine of DNA such that the detector ligand binds to any target DNA on the support; and

(e) detecting binding of the detector ligand to the support such that the degre or amount of binding is indicative of the extent of methylation of the target DNA.

Preferably, the capture ligand is selected from peptide nucleic acid (PNA) probe, oligonucleotide, modified oligonucleotide, singl stranded DNA, RNA, aptamer, antibody, protein, peptide, a combination thereof, or chimeric v rsions thereof.

More preferably, the capture ligand is a PNA probe or an oligonucleotide probe. Even more preferably, the capture ligand is a PNA probe.

The support can be any suitable support such as a plastic materials, fluorescent beads, magnetic beads, synthetic or natural membranes, latex beads, polystyrene, column supports, glass beads or slides, nanotubes, fibres or other organic or inorganic supports. Preferably, the support is a magnetic bead or a fluorescent bead.

The solid substrate is typically glass or a polymer, the most commonly used polymers being cellulose, polyacrylamide, nylon, polystyrene, polyvinyl chloride or polypropylene. The solid supports may be in the form of tubes, beads, discs or microplates, or any other surface suitable for conducting an assay. The binding processes are well-known in the art and generally consist of cross-linking covalently binding or physically adsorbing the molecule to the insoluble carrier. In a preferred form, step (b) comprises a plurality capture ligands arrayed on a solid support. The array may contain multiple copies of the same ligand so as to capture the same target DNA on the array or may contain a plurality of different ligands targeted to different DNA so as to capture a plurality of target DNA molecules on the array. Typically, the array contains from about 10 to 10,000 capture ligands. In one form, the array has less than about 500 capture ligands. It will be appreciated, however, that the array can have any number of capture ligands.

In one form, capture oligonucleotide probes or capture PNA probes can be placed on an array and used to capture bisulfite-treated DNA to measure methylated states of DNA. Array technology is well known and has been used to detect the presence of genes or nucleotide sequences in untreated samples. The present invention, how v r, can extend the usefulness of array technology to provide valuable information on methylation states of many different sources of DNA.

The sample can be any biological sample such as blood, urine. fa ces, semen, cerebrospinal fluid, cells or tissue such as brain, colon, urogenital, lung, renal, hematopoietic, breast, thymus, testis, ovary, or uterus, environmental samples, microorganisms including bacteria, virus, fungi, protozoan, viroid and the like.

In preferred forms, the sample is blood, colorectal tissue, brain or prostate tissue.

Preferably, the modifying agent is capable of modifying unmethylated cytosine but not methylated cytosine. The agent is preferably is selected from bisulfite, acetate and citrate. Preferably, the agent is sodium bisulfite and cytosine is modified to uracil.

The term “modifies” as used herein means the conversion of an unmethylated cytosine to another nucleotide which will distinguish the unmethylated from the methylated cytosine. Preferably, the agent modifies unmethylated cytosine to uracil. Preferably, the agent used for modifying unmethylated cytosine is sodium bisulfite, however, other agents that similarly modify unmethylated cytosine, but not methylated cytosine can also be used in the method of the invention. Sodium bisulfite (NaHSO₃) reacts readily with the 5,6-double bond of cytosine, but poorly with methylated cytosine. Cytosine reacts with the bisulfite ion to form a sulfonated cytosine reaction intermediate which is susceptible to deamination, giving rise to a sulfonated uracil. The sulfonate group can be removed under alkaline conditions, resulting in the formation of uracil. Thus all unmethylated cytosines will be converted to uracil while methylated cytosines will be protected from conversion so that ligands can be prepared that will recognise sequences containing cytosine or corresponding sequences containing uracil. The ratio of binding of the two probes can provide an accurate measure of the degree of methylation in a given DNA. Importantly, there is no need to amplify the DNA to obtain the required information thus overcoming potential errors and resulting in a faster and more simple assay amenable to automation.

In a pref rred form, the d tector ligand is directed to a CpG- or CNG-containing region of DNA, where N designates any one of the four possible bases A, T, C, or G. Preferably, the CpG- or CNG-containing region of DNA is in a regulatory region of a gene or an enhancer of any regulatory element or region. This region includes promoter, enhancer, oncogene, or other regulatory element which activity is altered by environmental factors including chemicals, toxins, drugs, radiation, synthetic or natural compounds and microorganisms or other infectious agents such as viruses, bacteria, fungi and prions. For example, the promoter or regulatory element can be a tumour suppressor gene promoter, oncogene or any other element that may control or influence one or more genes implicated in a disease state or changing normal state such as aging.

The presence of methylated CpG- or CNG-containing region of DNA in a specimen can be indicative of a cell proliferative disorder. The disorder can include low grade astrocytoma, anaplastic astrocytoma, glioblastoma, medulloblastoma, colon cancer, lung cancer, renal cancer, leukemia, breast cancer, prostate cancer, endometrial cancer and neuroblastoma.

In order to assist in the reaction of the DNA modifying agent optional additives such as urea, methoxyamine and mixtures thereof can be added.

Step (b) is typically used to capture a DNA of interest which will be analysed for methylation in subsequent steps of the method. Often a sample will contain genomic DNA from a cell source and that only one or a few genes will be of interest. Thus, step (b) allows the capture and concentration of DNA of interest. Preferably a first PNA or oligonucleotide probe is used in step (b).

In one preferred form, step (b) comprises a plurality of capture ligands arrayed on a solid support. The array may contain multiple copies of the same ligand so as to capture the same target DNA on the array for subsequent testing. Alternatively, the array may contain a plurality of different capture ligands targeted to different DNA molecules so as to capture many different target DNA samples on the array for subsequent testing. In one preferred form, the capture ligands are oligonucleotides or PNA molecules.

In step (d), two detector ligands can be used where one ligand is capable of binding to a region of DNA that contains one or more m thylated cytosines and the second ligand is capable of binding to a corresponding region of DNA that contains no methylated cytosines. A sampl can contain many copies of a target DNA with the copies having different amounts of methylation. Accordingly, the ratio of binding of the two ligands will be proportional to the degree of methylation of that DNA target in the sample. The two ligands can be added together in the one test or can be added in separate duplicate tests. Each ligand can have an unique marker which can be detected concurrently or separately in the one test or have the same marker and detected individually in separate tests.

In order to detect binding of the detector ligand to a target DNA, preferably the ligand has a detectable label attached thereto. The presence of bound label being indicative of the extent of binding of the ligand. Suitable labels include fluorescence, radioactivity, enzyme, hapten and dendrimer.

The detector ligands used in the invention for detecting CpG- or CNG-containing DNA in a sample, after bisulfite modification, can specifically distinguish between untreated DNA, methylated, and unmethylated DNA. Detector ligands in the form of oligonucleotide or PNA probes for the non-methylated DNA preferably have a T or A in the 3′ CG or CNG pair to distinguish it from the C retained in methylated DNA.

The probes of the invention were designed to be “substantially” complementary to one strand of the genomic locus to be tested and include the appropriate G or C nucleotides. This means that the primers must be sufficiently complementary to hybridize with a respective region of interest under conditions which allow binding. In other words, the probes should have sufficient complementarity with the 5′ and 3′ flanking sequences to hybridize therewith.

The PNA probes of the invention may be prepared using any suitable method known to the art. Typically, the PNA probes were prepared according to methods outlined in U.S. Pat. No. 6,110,676 (Coull et al 2000), incorporated herein by reference

The methods according to the present invention relating to methylation states of target DNA can use any DNA sample, in purified or unpurified form, as the starting material, provided it contains, or is suspected of containing, the specific DNA sequenc containing the target region (usually CpG or CNG). Typically, unamplified samples are used in the methods according to the present invention.

Th DNA-containing specimen used for detection of m thylated CpG or CNG may be from any source and may be extracted by a variety of techniques such as that described by Maniatis, et al (Molecular Cloning: A Laboratory Manual, Cold Spring Harbor, N.Y., pp 280, 281, 1982).

Where the DNA in the sample contains two strands, it is necessary to separate the strands of the DNA before it can be modified. Strand separation can be effected either as a separate step or simultaneously with chemical treatment. This strand separation can be accomplished using various suitable denaturing conditions, including physical, chemical, or enzymatic means, the word “denaturing” includes all such means. One physical method of separating DNA strands involves heating the DNA until it is denatured. Typical heat denaturation may involve temperatures ranging from about 80° to 105° C. for times ranging from about 1 to 10 minutes. Strand separation may also be induced by an enzyme from the class of enzymes known as helicases or by the enzyme RecA, which has helicase activity, and in the presence of riboATP, is known to denature DNA. The reaction conditions suitable for strand separation of DNA with helicases were described by Kuhn Hoffmann-Berling (CSH-Quantitative Biology, 43:63, 1978) and techniques for using RecA were reviewed in C. Radding (Ann. Rev. Genetics, 16:405437, 1982.

The detectable label may be fluorescent, or radioactive or contain a second label or marker in the form of a microsphere. The fluorescent or radioactive microsphere may be covalently bound to the capture or detector ligand.

In the case of a capture PNA ligand, the DNA binding can be detected via the phosphate groups thereby ensuring highly specific binding to the DNA and not to the negatively charged ligand or uncharged PNA.

The reagent is preferably a cationic molecule which binds to the DNA electrostatically. The detectable label attached thereto may be a fluorescent or radioactive molecule.

Preferably the specificity of hybridization to target DNA is used to discriminate between methylated cytosines and unm thylated cytosines.

The present invention makes particular use of the fact that PNA molecules have no net electrical charge while DNA, becaus of its phosphate backbone, are highly negatively charged. Detection of bound PNA probes can be a simpl molecule such as a positively charged fluorochrome, multiple molecules of which will bind specifically to the DNA in proportion to its length and can be directly detected. Many suitable fluorochromes that bind to DNA, some selective for single-stranded DNA, and that differ in their excitation and emission wavelengths were known. The detection system could also be an enzyme carrying a positively charged region that will selectively bind to the DNA and that can be detected using an enzymatic assay, or a positively charged radioactive molecule that binds selectively to the captured DNA.

Using PNA probes as one of the ligands in this procedure has very significant advantages over the use of oligonucleotide probes. PNA binding reaches equilibrium faster and exhibits greater sequence specificity and, as PNAs are uncharged, they bind the target DNA molecules with a higher binding coefficient.

As the invention can use direct detection methods, they give a true and accurate measure of the amount of a target DNA in a sample. The methods were not confounded by potential bias inherent in methods that rely for signal amplification on processes such as PCR, where the enzymes commonly used in such procedures can introduce systematic bias through differential rates of amplification of different sequences.

In a fifth aspect, the present invention provides a method for detecting a methylated CpG- or CNG-containing DNA, the method comprising:

A method for detecting a methylated CpG- or CNG-containing DNA, the method comprising:

(a) treating a sample containing DNA with bisulfite to modify unmethylated cytosine to uracil in the DNA;

(b) providing to the treated sample a detector PNA ligand capable of distinguishing between methylated and unmethylated cytosine of DNA; and

(c) detecting the methylated DNA based on the presence or abs nce of binding of the detector PNA ligand.

In one pref rred from, the method comprises:

(a) treating a DNA-containing specimen with bisulfite to modify unmethylated cytosine to uracil,

(b) providing to the treated sample a detector ligand capable of binding to a methylated CpG- or CNG-containing DNA but not to a corresponding unmethylated CpG- or CNG-containing DNA; and

(c) detecting binding of the ligand to DNA in the sample such that binding is indicative of methylation of the DNA.

Preferably, the detector ligand is a peptide nucleic acid (PNA) probe.

In a preferred from, the invention provides a method for estimating extent of methylation of a target DNA in a sample, the method comprising:

(a) treating a sample containing DNA with bisulfite to modify unmethylated cytosine to uracil;

(b) providing a solid support in the form of a magnetic bead to which is. bound a capture PNA or oligonucleotide ligand which is capable of recognising a first part of a target DNA sequence;

(c) contacting the support with the treated sample suspected of containing the target DNA such that target DNA in the sample binds to the support via the capture ligand;

(d) contacting the support with a detector PNA ligand capable of distinguishing between methylated and unmethylated cytosine of DNA; and

(e) determining the extent of methylation of the DNA bound to the support by measuring the amount of bound detector ligand.

In a sixth aspect, the present invention relates to use of an agent that modifies unmethylated cytosine but not methylated cytosine and one or more ligands, preferably on or more peptide nucleic acid (PNA) problems, capable of distinguishing between m thylated and unmethylated cytosine of DNA in methods for assaying methylation of target DNA.

There as a number of detector systems and instruments available for detecting or measuring fluorescence or radioactivity. Improvements and advancement in instrumentation is being made by a number of manufacturers. It will be appreciated that many different measuring instruments can be used for the present invention. For example, Multi Photon Detection is a proprietary system for the detection of ultra low amounts of selected radioisotopes. It is 1000 fold more sensitive than existing methods. It has a sensitivity of 1000 atoms of iodine 125, with quantitation of zeptomole amounts of biomaterials. It requires less than 1 picoCurie of isotope which is 100 times less activity than in a glass of water. A family of MPD instruments already exists for measuring radioactivity in a sample. They consist of instruments that are configured for 96 well, 384 well and higher. MPD uses coincident multichannel detection of photons coupled with computer controlled electronics to selectively count only those photons that are compatible with an operator-selected radioisotope. As many different isotopes can be used, this is a multicolor system. The MPD imager system is at least 100 fold more sensitive than a phosphor imager. Such instrumentation would be particularly suitable in the detection part of the present invention where ligands or supports are made radioactive.

Beads containing capture or detector ligands bound thereto can be processed or measured by cell sorters which measure fluorescence. Examples or suitable instruments include flow cytometers and modified versions thereof.

The methods according to the present invention are particularly suitable for scaling up and automation for processing many samples.

Notwithstanding the above, the methods described can be used in conjunction with such amplification procedures if it is necessary to amplify limiting amounts of DNA in order provide enough material for detection.

Methylated DNA: In a particular adaptation as detailed in the present invention, the methods can be used to distinguish the presence of methylated cytosines in DNA that has been treated with sodium bisulfite. As cytosines were converted to uracils while methyl cytosines remain unreacted, the s quence of bisulfite-treated DNA derived from methylated and unmethylated molecules is different. By choosing a specific PNA ligand (5 to 40 residues long, preferably 15±5 residues long) to selected target regions the specificity of hybridisation can be used to discriminate between methylated cytosines at CpG or CNG sites (which remain as cytosines) and unmethylated CpG or CNG sites where the cytosine is converted to uracil, while ensuring that only molecules in which cytosines that were not in CpG or CNG sites have fully reacted and been converted to uracils were assessed.

Methylated cytosines at other sites can similarly be detected. Appropriate PNA probes can be used as controls to identify the presence of molecules that. have not reacted completely with bisulfite (one or more cytosines not converted to uracil). It will be appreciated, however, that other ligands which can differentiate between the methylation states of DNA can be used in a similar manner.

The methods were amenable for use in a variety of formats including multiwell plates, micro-arrays and particles in suspension. The appropriate selection of specific ligands for use in an array format can allow for the simultaneous determination of the methylation state of individual cytosines in multiple target regions.

Polymorphism/mutation detection: The methods according to the present invention can be applied to the discrimination of mutant alleles of a gene where the sequence of the capture ligand and/or the detector ligand will match with one allele but mismatch with the other.

DNA Quantification: By using the methods according to the present invention, it is possible to directly determine within a DNA population the proportion of molecules having one sequence versus another at a particular region. This can be done by coupling ligands representing the alternate forms of the sequence to supports such as microspheres charged with different fluorochromes or radioactive molecules. Such differences in sequence may be differences in the original base sequence of the gene or differences in base sequence in bisulfite-treated DNA that were due to differences in methylation in the original DNA.

Cell quantification: The methods can be applied to determining the ratio of cells in a population (such as in cancer and normal cells) that differ in base sequence at a particular site in the genome.

Variations: The methods were amenable for use in a variety of formats including multiwell plates, micro-arrays and particles in suspension. The appropriate selection of specific PNA probes for use in an array format can allow for the simultaneous determination of the presence of different DNA sequences, eg. for the determination of the methylation state of individual cytosines in multiple target regions.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element; integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

In order that the present invention may be more clearly understood, preferred forms will be described with reference to the following drawings and examples.

### BRIEF DESCRIPTION OF THE DRAWINGS

FIG. 1 shows a general overview of sandwich signal amplification methodology using PNA probes for detection of methylated DNA.

FIG. 2 hows a general overview of sandwich signal amplification methodology using PNA probes and magnetic beads for detection of methylated DNA.

FIG. 3 shows part of the nucleic acid sequence of the GSTP1 gene and ten PNA prob s useful for detecting various methylation states of that gene region.

FIG. 4 shows a comparison of the effect of microsphere bead size on hybridisation signal.

FIG. 5 shows detection capabilities for prostate cancer cell line and tissue DNA extracts using PNA technology and methods of the invention.

FIG. 6 shows effect of PNA concentration on sensitivity of method using ligands bound to micotitre well plates.

FIG. 7 shows results of single methylation using Oligreen detection agent.

FIG. 8 shows results of detection of methylated DNA sequences in a background of unmethylated sequences.

FIG. 9 shows results of detection of unmethylated DNA sequences in a background of methylated sequences.

FIG. 10 shows an example of the methylation pattern of GSTP1 in prostate cancer.

### MODE(s) FOR CARRYING OUT THE INVENTION

Methylation

The amount or degree of methylation of genomic DNA has implications in many conditions such as aging, genetic abnormalities, cancer and other disease states. A number of important implications of methylation states were set out below.

The fusion of Embryonic Stem Cells with adult thymocytes to examine the reprogramming that occurs at the level of DNA methylation after the fusion has been made. The inactive somatic X becomes activated as visualized by whole chromosome examination (Tada et al., 2001; Current Biology, 11, 1553-1558).

Examination of methylation patterns in specific DNA regions in the clinicopathological features of sporadic colorectal cancers, as an inexpensive and accurate way of identifying such tumors (Ward et al., 2001; Gut, 48,821-829), and the methylation patterns in stem cells in human colon crypts (Ro et al., 2001, Proc Natl Acad Sci, USA, 98, 10519-10521; Yatabe etal., 2001, Proc. Natl. Acad Sci USA, on line edition).

Methylation patterns in prostate cancer, and in c II lines treated with 5-azacytidine in order to reactivate specific genes (Chetcuti et al., 2001, Cancer Research, 61,6331-6334).

Methylation patterns in the various Estrogen receptors in uterus endometrial cancers where gene inactivation via methylation occurs in many cancers but is not at a high frequency in normal individuals (Sasaki et al., 2001, Cancer Research, 61, 3262-3266).

Methylation patterns in bladder cancer (Markl et al., 2001, Cancer Research, 61, 5875-5884).

Methylation patterns in breast cancer (Nielsen et al., 2001, Cancer Letters, 163, 59-69).

Methylation patterns in specific promoters involved in lung and breast cancers (Burbee et al., 2001, J Natl Cancer Institute, 93, 691-699).

Methylation patterns in free DNA in the plasma of patients with esophageal adenocarcinomas (Kawakami et al., 2000, J Natl Cancer Institute, 92, 1805-1811).

Methylation of the CDH1 promoter in hereditary diffuse gastric cancer (Grady et al., 2000, Nature Genetics, 26,16-17).

Genomic imprinting, in which, for example, a paternal allele of a gene is active, and the maternal allele is inactive, or vice versa. This inactivation is accomplished via methylation changes in the genes involved, or in sequences nearby to them. In essence, DNA regions become methylated in the germ line of one sex, but not in that of another (Mann, 2001, Stem Cells, 19, 287-294).

Genome-wide methylation patterns in studies of cloning of various species (sheep, cattle, goats, pigs and mice), via nuclear transfer or in vitro fertilization. Thus the methylation patterns of donor nuclei that were inserted into oocyt s vary greatly, and this is thought to be the reason why there is such a high failure rate in current cloning experiments. These differentiated nuclei probably require more reprogramming that less differentiated ones such as in Embryonic Stem Cells (Kang et al, 2001; Nature Genetics, 28, 173-177; Humphreys et al., 2001, Science, 293, 95-97).

Excessive hyper-methylation patterns in 24 cancer cell lines versus normal tissues (Smiraglia et al., 2001, Human Molecular Genetics, 10, 1413-1419).

Insertion of methylated DNA into a non methylated mini gene construct to examine the effects on gene expression and imprinting (Holmgren et al., 2001, Current Biology, 11, 1128-1130).

Methylation patterns in mature B cell lymphomas, where specific genes were inactivated by methylation (Malone et al., 2001, Proc Natl Acad Sci USA 98, 10404-10409).

Methylation patterns of particular genes in acute myeloid leukemia (Melki et al., 1999, Leukemia, 13, 877-883).

Analysis of the Mecp2 gene in knockout mice. This protein is involved in binding to methylated sites in DNA and is thought to be involved in Rett syndrome, which is an inherited neurological disorder (Guy et al., Nature Genetics, 27, 322-326).

Methylation patterns of 5 specific genes during the normal aging process, and in ulcerative colitis (Issa et al., 2001, Cancer Research, 61, 3573-3577).

Loss of methylation in the processes of apoptosis, which impinge upon signal transduction pathways, cell cycle control, movement of mobile elements within the genome (Jackson-Grusby et al., 2001, Nature Genetics, 27, 31-39).

Comparison of the methylation patterns of promoter and gene regions in different species, such as human and mouse, to determine the evolutionary conservation or lack thereof of CpG islands involved in gene regulation (Cuadrado et al., 2001, EMBO Reports, 21, 586-592).

DNA methylation patterns in testicular sperm at different developmental stages (Manning et al., 2001, Urol Int, 67, 151-155).

Immuno histochemical staining using a monoclonal antibody to analyze DNA methylation patterns (Piyathilake et al., 2000, Biotechnic and Histochem, 75, 251-258).

Differences between the methylation patterns of genes and pseudogenes (Grunauet al., 2000, Human Mol Genet, 9, 2651-2663).

5-methylycytosine content of model invertebrates such as Drosophila melanogaster (Gowher et al., 2000, EMBO J, 19, 6918-6923).

Large scale mapping of human promoters using the methylation patterns of CpG islands (Ioshikhes et al, 2000, Nature Genetics, 26, 61-63).

Induced changes in the processes of chromatin remodelling, DNA methylation and gene expression during mammalian development due to changes in the expression of the ATRX gene which give rise to mental retardation, facial dysmorphism, urogenital abnormalities and alpha thalassemia (Gibbons et al., 2000, Nature Genetics, 24, 368-371).

Boundaries between methylated and unmethylated domains in the promoter region of the GSTP1 gene involved in prostate cancer (Millar et al., 2000, J Biological Chemistry, 275, 24893-24899; Millar et al., 1999, Oncogene, 18, 1313-1324).

Methylation changes during the normal processes of aging (Toyota et al., 1999, Seminars in Cancer Biology, 9, 349-357).

Methylation changes in aging and in atherosclerosis in the cardiovascular system, (Post et al., 1999, Cardiovascular Research, 43, 985-991) and during normal aging and cancers in colorectal mucosa (Ahuja et al., 1998, Cancer Research, 58, 5489-5494).

Methylation patterns in germ cells and sertoli cells in testis (Coffigny et al., 1999, Cytogenet Cell Genets, 87, 175-181).

DNA methylation changes during the development of model vertebrates such as the zebrafish (Macleod et al., 1999, Nature Genetics, 23, 139-140).

Methylation patterns in the promoter regions of the human histo-blood ABO genes (Kominato et al., 1999, J Biol Chem, 274, 37240-37250).

Methylation patterns during mammalian preimplantation development using monoclonal antibodies (Rougier et al., 1999, Genes and Development, 12, 2108-2113).

Methylation patterns induced by various cancer chemotherapeutic drugs (Nyce, 1997, Mutation Research, 386, 153-161; Nyce 1989, Cancer Research, 49, 5829-5836) and the changes in DNA methylation in phenobarbital-induc d and spontaneous liver tumors (Ray et al., 1994, Molecular Carcinogenesis 9, 155-166).

Analysis of 5-methycytosine residues in DNA by the bisulfite sequencing method (Grigg, 1996, DNA Sequence, 6, 189-198).

Isolation of CpG islands using a methylated DNA binding column (Cross et al., 1994, Nature Genetics, 6, 236-244).

Is KSHV lytic growth induced by a methylation-sensitive switch? (Laman and Boshoff, Trends Microbiol 2001 October; 9(10):464-6). Both latent and lytic growth of Kaposi's sarcoma-associated herpesvirus (KSHV or HHV-8) contribute to its pathogenesis.

As can be seen from the large number of examples of different methylation states and implications provided above, it will be appreciated that the present invention offers a powerful tool for the study of methylation and thus is useful for many aspects of disease and health.

Table 1 shows some examples of solid supports useful for attaching capture ligands of the present invention. Table 2 shows possible choices of detector systems for use in the present invention.

TABLE 1
Solid supports for attachment of capture ligands

| | fluoro | | magnetic | latex | p/styrene | mem- | |
|---|---|---|---|---|---|---|---|
| label | bead | column | bead | bead | bead | brane | glass |
| PNA | + | + | + | + | + | + | + |
| Oligo | + | + | + | + | + | + | + |
| RNA | + | + | + | + | + | + | + |
| Hybrid | + | + | + | + | + | + | + |

TABLE 2
Detection systems for detection ligands

| | Fluoro | Magnetic | latex | p/styrene | | |
|---|---|---|---|---|---|---|
| Lab I | Bead | bead | bead | bead | Glass | Aptamer |
| Pre-label | + | | | | | |
| Florescence | + | + | + | + | + | + |
| radiolabel | + | + | + | + | + | + |
| Dendrimer | + | + | + | + | + | + |

Materials and Methods

Peptide Nucleic Acids (PNAS)

Peptide Nucleic Acids (PNAs) are non-naturally occurring polyamides which can hybridize to nucleic acids (DNA and RNA) with sequence specificity. (See U.S. Pat. No. 5,539,082 and Egholm et al., Nature (1993) 365, 566-568). PNA's are candidates as alternatives/substitutes to nucleic acid probes in probe-base hybridization assays because they exhibit several desirable properties. PNA's are achiral polymers which hybridize to nucleic acids to form hybrids which are more thermodynamically stable than a corresponding nucleic acid/nucleic acid complex (See: Egholm et. al., Nature (1993) 365, 566-568). Being non-naturally occurring molecules, they are not known to be substrates for the enzymes which are known to degrade peptides or nucleic acids. Therefore, PNA's should be stable in biological samples, as well as, have a long shelf-life. Unlike nucleic acid hybridization which is very dependent on ionic strength, the hybridization of a PNA with a nucleic acid is fairly independent of ionic strength and is favoured at low ionic strength under conditions which strongly disfavour the hybridization of nucleic acid to nucleic acid (See: Egholm et. al., Nature, p. 567). The effect of ionic strength on the stability and conformation of PNA complexes has been ext nsively investigated (See: Tomac et al. J. Am. Chem. Soc. (1996) 118, 5544-5552). Sequence discrimination is more efficient for PNA recognizing DNA than for DNA recognizing DNA (See: Egholm et al., Nature, p. 566). However, the advantages in point mutation discrimination with PNA probes, as compared with DNA probes, in a hybridization assay appears to be somewhat sequence dependent (See: Nielsen et al. Anti-Cancer Drug Design (1993) 8, 53-65). As an additional advantage, PNA's hybridize to nucleic acid in both a parallel and antiparallel orientation, though the antiparallel orientation is preferred (See: Egholm et al., Nature, p. 566).

PNAs are synthesized by adaptation of standard peptide synthesis procedures in a format which is now commercially available. (For a general review of the preparation of PNA monomers and oligomers. please see: Dueholm et al., New J. Chem. (1997), 21, 19-31 or Hyrup et: al., Bioorganic & Med. Chem. (1996) 4, 5-23). Labelled and unlabelled PNA oligomers can be purchased (See: PerSeptive Biosystems Promotional Literature: BioConcepts, Publication No. NL612, Practical PNA, Review and Practical PNA, Vol. 1, Iss. 2) or prepared using the commercially available products.

There are indeed many differences between PNA probes and standard nucleic acid probes. These differences can be conveniently broken down into biological, structural, and physico-chemical differences. As discussed above and below, these biological, structural, and physico-chemical differences may lead to unpredictable results when attempting to use PNA probes in applications were nucleic acids have typically been employed. This non-equivalency of differing compositions is often observed in the chemical arts.

With regard to biological differences, nucleic acids are biological materials that play a central role in the life of living species as agents of genetic transmission and expression. Their in vivo properties are fairly well understood. PNA, however, is a recently developed totally artificial molecule, conceived in the minds of chemists and made using synthetic organic chemistry. It has no known biological function.

Structurally, PNA also differs dramatically from nucleic acid. Although both can employ common nucleobases (A, C, G, T, and U), the backbones of these molecules are structurally diverse. Th backbones of RNA and DNA are composed of repeating phosphodiester ribose and 2-deoxyribos units. In contrast, the backbones of PNA are composed on N-(2-aminoethyl)glycine units. Additionally, in PNA the nucleobases are connected to the backbone by an additional methylene carbonyl unit.

D spite its name, PNA is not an acid and contains no charged acidic groups such as those present in DNA and RNA. Because they lack formal charge, PNAs are generally more hydrophobic than their equivalent nucleic acid molecules. The hydrophobic character of PNA allows for the possibility of non-specific (hydrophobic/hydrophobic interactions) interactions not observed with nucleic acids. Furthermore, PNA is achiral, providing it with the capability of adopting structural conformations the equivalent of which do not exist in the RNA/DNA realm.

The physico/chemical differences between PNA and DNA or RNA are also substantial. PNA binds to its complementary nucleic acid more rapidly than nucleic acid probes bind to the same target sequence. This behaviour is believed to be, at least partially, due to the fact that PNA lacks charge on its backbone. Additionally, recent publications demonstrate that the incorporation of positively charged groups into PNAs will improve the kinetics of hybridization. (See: lyer et al. J. Biol. Chem. (1995) 270, 14712-14717). Because it lacks charge on the backbone, the stability of the PNA/nucleic acid complex is higher than that of an analogous DNA/DNA or RNA/DNA complex. In certain situations, PNA will form highly stable triple helical complexes or form small loops through a process called “strand displacement”. No equivalent strand displacement processes or structures are known in the DNA/RNA world.

In summary, because PNAs hybridize to nucleic acids with sequence specificity, PNAs are useful candidates for developing probe-based assays. However, PNA probes are not the equivalent of nucleic acid probes. Nonetheless, even under the most stringent conditions both the exact target sequence and a closely related sequence (e.g. a non-target sequence having a single point mutation (single base pair mismatch)) will often exhibit detectable interaction with a labelled nucleic acid or labelled PNA probe (See: Nielsen et al. Anti-Cancer Drug Design at p. 56-57 and Weiler et al. at p. 2798, second full paragraph). Any hybridization to a closely related non-target sequence will result in the generation of undesired background signal. Because the sequ nces are so closely related, point mutations are some of the most difficult of all nucleic acid modifications to detect using a probe-based assay. Numerous diseases, such as sickle cell anemia and cystic fibrosis, are caused by a single point mutation of genomic nucleic acid. Consequently, any method, kits or compositions which could improve the specificity, sensitivity and reliability of probe-based assays would be useful in the detection, analysis and quantitation of DNA containing samples.

Sodium Bisulfite—A Specific Deamination Method

Methods for treating nucleic acid with sodium bisufite can be found in a number of references including Frommer et al 1992, Proc Natl Acad Sci 89:1827-1831; Grigg and Clark 1994 BioAssays 16:431-436; Shapiro et al 1970, J Amer Chem Soc 92:422 to 423; Wataya and Hayatsu 1972, Biochemistry 11:3583-3588.

Detection Systems

Coating Magnetic Beads

The PNA used for attachment to the magnetic beads can be modified in a number of ways. In this example, the PNA contained either a 5′ or 3′ amino group for the covalent attachment of the PNA to the beads using a heterobifunctional linker such as is used EDC. However, the PNA can also be modified with 5′ groups such as biotin which can then be passively attached to magnetic beads modified with avidin or steptavidin groups.

Ten μL of carboxylate modified Magnabind™ beads (Pierce) or 100 μL of Dynabeads™ Streptavidin (Dynal) were transferred to a clean 1.5 mL tube and 90 μL of PBS solution added to the magnetic beads.

The beads were mixed then magnetised and the supernatant discarded. The beads were washed ×2 in 100 μL of PBS per wash and finally resuspended in 90 μL of 50 mM MES buffer pH 4.5 or another buffer as determined by the manufactures' specifications.

One μL of 250 μM PNA (concentration dependant on the specific activity of the sel cted PNA as determined by oligonucleotide hybridisation experiments) is added to the sample and the tube vortexed and left at room temperatur for 10-20 minutes.

Ten μL of a freshly prepared 25 mg/mL EDC solution (Pierce/Sigma) is then added, the sample vortexed and incubated at either room temperature or 4° C. for up to 60 minutes.

The samples were then magnetised, the supernatant discarded and the beads, if necessary, be blocked by the addition of 100 μL either 0.25 M NaOH or 0.5 M Tris pH 8.0 for 10 minutes.

The beads were then washed ×2 with PBS solution and finally resuspended in 100 μl PBS solution.

Hybridisation Using the Magnetic Beads

Ten μL of PNA coated Magnabind™ beads were transferred to a clean tube and 40 μL of either ExpressHyb™ buffer (Clontech) either neat or diluted 1:1 in distilled water or any other commercial or in-house hybridization buffer. The buffers may also contain either cationic/anionic or zwittergents at known concentration or other additives such as Heparin and poly amino acids.

Heat denatured sample of DNA 1-5 μL was then added to the above solution and the tubes vortexed and then incubated at 55° C. or another temperature depending on the melting temperature of the chosen PNA for 20-60 minutes.

The samples were magnetised and the supernatant discarded and the beads washed ×2 with 0.1×SSC/0.1% SDS at the hybridisation temperature from earlier step for 5 minutes per wash, magnetising the samples between washes.

Dual PNA Capture

PNA#1 was coupled to a carboxylate modified magnetic bead via a N- or C-terminal amine of the PNA and washed to remove unbound PNA.

The PNA/bead complex is then hybridised to the target DNA in solution using appropriate hybridisation and washing conditions.

The target DNA was then released from the magnetic bead using appropriate methods and transferred to a tube containing a second PNA/magnetic beads complex targeted to the opposite end of the DNA molecule.

The second PNA/bead complex or oligo/bead complex was then hybridised to the target DNA in solution using appropriate hybridisation and washing conditions.

A third PNA or oligonucleotide complementary to the central region of the target DNA could be used as a detector molecule. This detector molecule can be labelled in a number of ways.

(i) The PNA or oligonucleotide can be directly labelled with a radioactive isotope such as P³² or I¹²⁵ and then hybridised with the target DNA.

(ii) The PNA or oligonucleotide can be labelled With a fluorescent molecule such as Cy-3 or Cy-5 and then hybridised with the target DNA:

(iii) An amine modified PNA or oligonucleotide can be labelled in either of the above ways then coupled to a carboxylate modified microsphere of known size then the sphere washed to remove unbound labelled PNA or oligo. This bead complex can then be used to produce a signal amplification system for the detection of the specific DNA molecule.

(iv) The PNA or oligonucleotide can be attached to a dendrimer molecule either labelled with fluorescent or radioactive groups and this complex used to produce a signal amplification.

(v) The PNA or oligonucleotide labelled in any of the above ways and hybridised to the target DNA on a solid support can be released into solution using a single stranded specific nuclease such a mung bean nuclease or S1 nuclease. The released detector molecule can be read in a flow cytometer like devic .

Preparation of Radio-Labelled Detector Spheres

A PNA or oligonucleotide molecule can be either 3′ or 5′ labelled with a molecule such as an amine group, thiol group or biotin.

The labelled molecule can also have a second label such as P³² or I¹²⁵ incorporated at the opposite end of the molecule to the first label.

This dual labelled detector molecule can be covalently coupled to a carboxylate or modified latex bead for example of known size using a hetero-bifunctional linker such as EDC. Other suitable substrates can also be used depending on the assay.

The unbound molecules can then be removed by washing leaving a bead coated with large numbers of specific detector/signal amplification molecules.

These beads can then be hybridised with the DNA sample of interest to produce signal amplification.

Preparation of Fluorescent Labelled Detector Spheres

A PNA or oligonucleotide molecule can be either 3′ or 5′ labelled with a molecule such as an amine group, thiol group or biotin.

The labelled molecule can also have a second label such as Cy-3 or Cy-5 incorporated at the opposite end of the molecule to the first label.

This dual labelled detector molecule can now be covalently coupled to a carboxylate or modified latex bead of known size using a hetero-bifunctional linker such as EDC.

The unbound molecules can then be removed by washing leaving a bead coated with large numbers of specific detector/signal amplification molecules.

These beads can then be hybridised with the DNA sample of interest to produce signal amplification.

Preparation of Enzyme Labelled Detector Spheres

A PNA or oligonucleotide molecule can be either 3′ or 5′ labelled with a molecule such as an amin group or a thiol group.

The labelled molecule can also have a second label such as biotin or other molecules such as horse-radish peroxidase or alkaline phosphatase conjugated on via a hetero-bifunctional linker at the opposite end of the molecule to the first label.

This dual labelled detector molecule can now be covalently coupled to a carboxylate or modified latex bead of known size using a hetero-bifunctional linker such as EDC.

The unbound molecules can then be removed by washing leaving a bead coated with large numbers of specific detector/signal amplification molecules.

These beads can then be hybridised with the DNA sample of interest to produce signal amplification.

Signal amplification can then be achieved by binding of a molecule such as streptavidin or an enzymatic reaction involving a colorimetric substrate.

PNA Oligomer Combinations

In all of the above cases the initial hybridization event involved the use of magnetic beads coated with a PNA complimentary to the DNA of interest.

The second hybridisation event can involve any of the methods mentioned above.

This hybridisation reaction can be done with either a second PNA complimentary to the DNA of interest or an oligonucleotide or modified oligonucleotide complementary to the DNA of interest. As fluorescent beads of convenient size in these assays, carry >10⁶ fluorochrome molecules and a single fluorescent bead can be detected readily, the method has the potential sensitivity to assay one or a few DNA molecules from on or a few cells.

Dendrimers and Aptamers

Dendrimers are branched tree-like molecules that can be chemically synth sised in a controlled manner so that multiple layers can be generated that were labelled with specific molecules. They were synthesised stepwise from the centre to the periphery or visa-versa.

One of the most important parameters governing dendrimer structure and its generation is the number of branches generated at each step; this determines the number of repetitive steps required to build the desired molecule.

Dendrimers can be synthesised that contain radioactive labels such as I¹²⁵ or P³² or fluorescent labels such as Cy-3 or Cy-5 to enhance signal amplification.

Alternatively dendrimers can be synthesised to contain carboxylate groups or any other reactive group that could be used to attach a modified PNA or DNA molecule.

Results

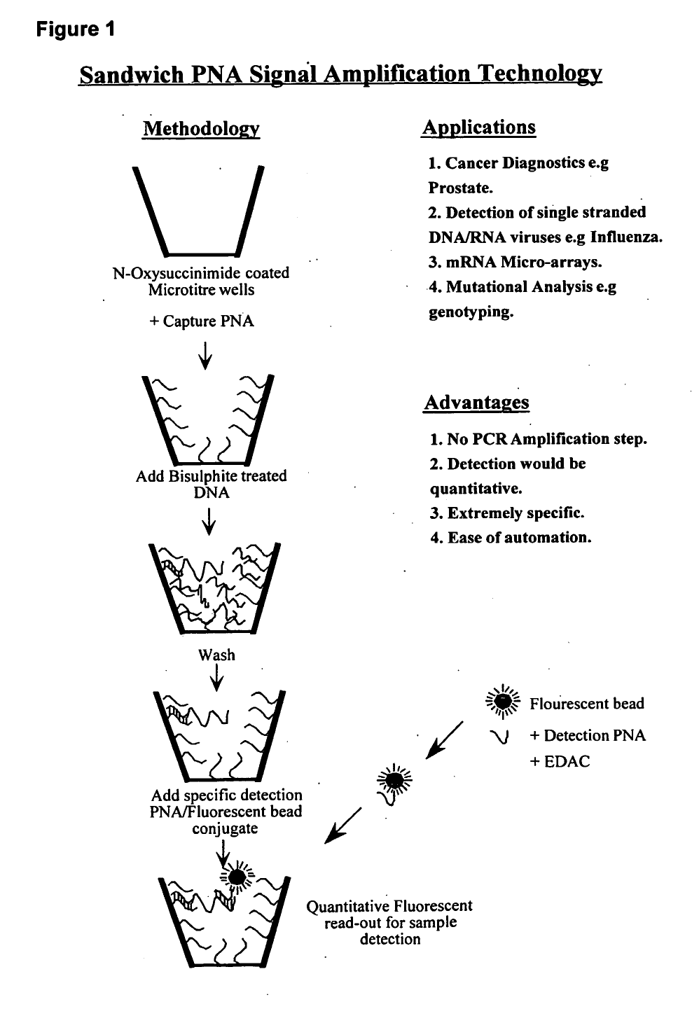

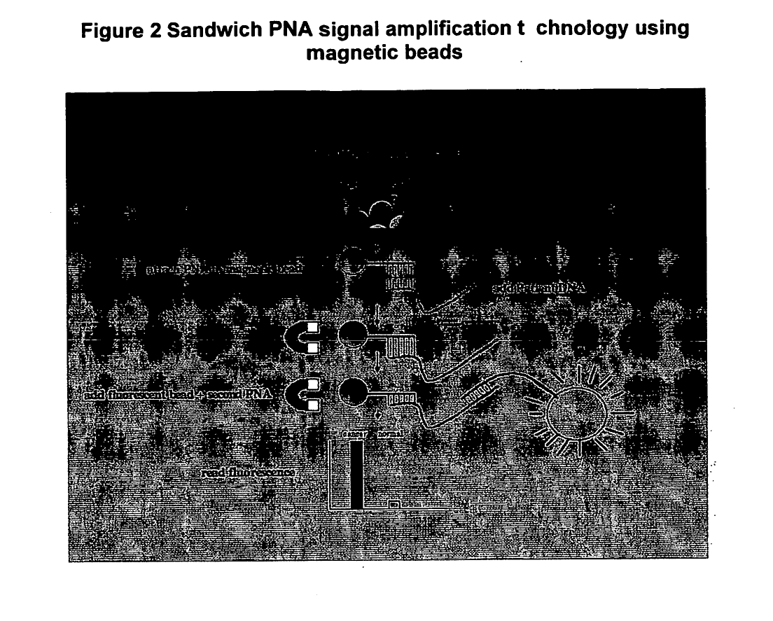

FIG. 1 and FIG. 2 show examples of the method of the invention using sandwich PNA signal amplification using solid supports and magnetic beads, respectively. Although PNA is exemplified as the ligand in FIG. 1 and FIG. 2, it will be appreciated that other capture or detector ligands such as oligonucleotides can be used in these methods.

A solid support in the form of a microfilter well was provided and coated with N-oxysuccinimide to assist in the adhesion of PNA or other ligand to the well.

A first PNA which is complementary to a first part of the target nucleotide sequence is added to the well and attached to this solid support.

Bisulfite treated DNA is then added to the well and allowed to hybridise with the PNA to capture the target DNA which has hybridised to the PNA and subsequently bound to the well.

The well is then washed to remove the hybridisation solution and any non-hybridised DNA leaving only the hybridised DNA captured on the w II.

Next a second PNA, which is complementary to a second part of the target nucleotide sequence is linked to microsphere beads having fluorescent labelling. The second linked PNA is then hybridised with the target DNA already bound to the well. The well is then washed to remove the unhybridized second PNA/microsphere complex leaving only the PNA/microsphere complex and fluorescent label associated with the target DNA sequence.

The fluorescence was then measured to determine the level of target DNA.

### EXAMPLES

I. Detection of Methylated Promoter Sequences of the GSTP1 Zone

The promoter region of the GSTP1 gene has been shown to be hypermethylated in prostate cancer (Lee W H, Morton R A, Epstein J I, Brooks J D, Campbell P A, Bova G S, Hsieh W-S, lsaacs W B and Nelson W G. Cytidine methylation of regulatory sequences near the pi-class glutathione S-transferase gene accompanies prostatic carcinogenesis. PNAS 91:11733-11737 (1994)). Bisulfite sequencing has defined region and specific CpG sites that were methylated in prostate cancer cells, but not in normal prostate (Millar D S, Ow K K, Paul C L, Russell P J, Molloy P L and Clark S J. Detailed methylation analysis of the glutathione S-transferase pi (GSTP1) gene in prostate cancer. Oncogene 18:1313-1324 (1999); Millar D S, Paul C, Molloy P L and S J Clark (2000). A distinct sequence (ATAAA)ₙ separates methylated and unmethylated domains at the 5′ end of the GSTP1 CpG island. J. Biol Chem. 275: 24893-24899). These studies define regions that provide good discrimination between DNA from normal tissues and DNA from prostate cancer cells, based on their different methylation pattern.

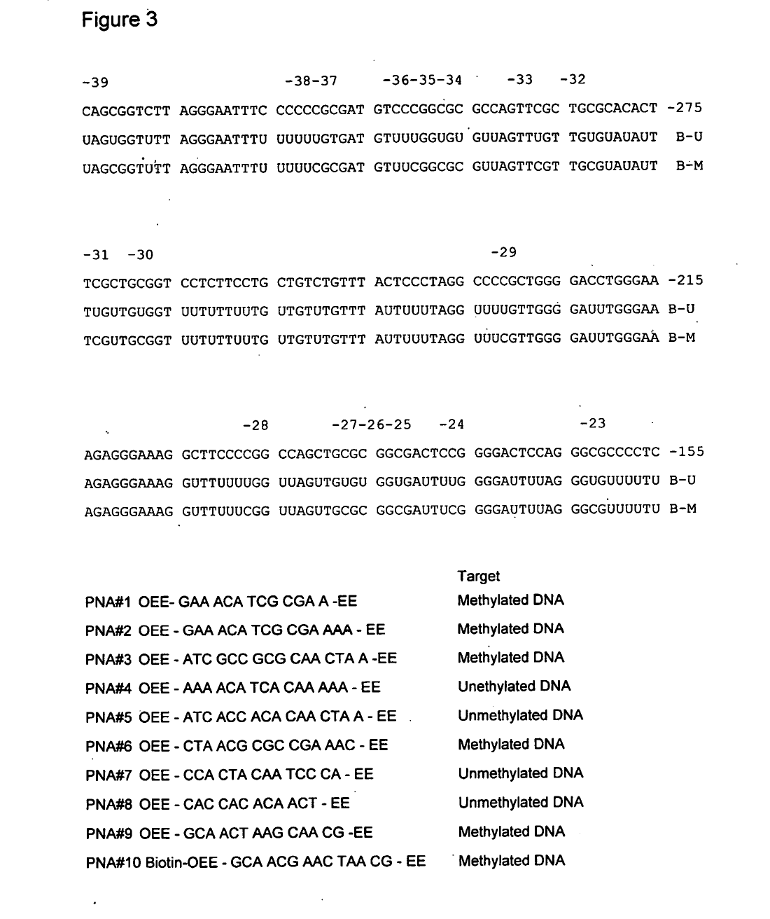

The sequence of part of this region of the GSTP1 gene is shown in FIG. 3. Individual CpG sites were numbered above the sequence relative to the position of the transcription start site. Base numbers relative to the transcription start site were shown at the ends of lines of sequence. The sequence corresponds to bases 981 to 1160 of Genbank Accession No. M24485 (with inclusion of an additional CG). The number of each CpG site is indicated abov each site. Also indicated above CpG site -33 is the sequence of th polymorphic variant (p) which occurs in this region. The top line of ach triplet shows the normal DNA sequence; the second lin , B-U, shows th s quence that would arise following bisulfite treatment of DNA that contained no methylated cytosin s (cytosines converted to uracils); the third line, B-C, shows the bisulfite-modified sequence produced if all cytosines at CpG sites were methylated. The position of PNAs #1 to #10 is shown under the sequence.

PNAs were synthesised that would hybridise to specific sites in the bisulfite-treated DNA as shown. Regions of sequence were chosen that contained a number of cytosines both within CpG sites (and potentially methylated) and not in CpG sites. PNAs were designed so that they will match perfectly if all cytosines at CpG sites in the DNA were methylated and hence had remained as cytosines and if all other cytosines had been efficiently converted to uracils. Thus, only properly bisulfite-converted, methylated DNA sequences should hybridise with the PNA probes under discriminating hybridisation conditions. The sequences of the ten PNA probes are shown below:

| | |
|---|---|
| PNA#1 | |
| P-Linker-GAA ACA TCG CGA A-NH₂ | SEQ ID NO:2 |
| PNA#2 | |
| P-Linker-GAA ACA TCG CGA AAA-NH₂ | SEQ ID NO:3 |
| PNA#3 | |
| P-Linker-ATC GCC GCG CAA CTA A-NH₂ | SEQ ID NO:4 |
| PNA#4 | |
| P-Linker-AAA ACA TCA CAA AAA -NH₂ | SEQ ID NO:5 |
| PNA#5 | |
| P-Linker-ATC ACC ACA CAA CTA A-NH₂ | SEQ ID NO:6 |
| PNA#6 | |
| P-Linker-CTA ACG CGC CGA AAC-NH₂ | SEQ ID NO:7 |
| PNA#7 | |
| P-Linker-CCA CTA CAA TCC CA-NH₂ | SEQ ID NO:8 |
| PNA#8 | |
| P-Linker-CAC CAC ACA ACT-NH₂ | SEQ ID NO:9 |
| PNA#9 | |
| P-Linker-GCA ACT AAG CAA CG-NH₂ | SEQ ID NO:10 |
| PNA#10 | |
| P-Linker-GCA ACG AAC TAA CG-NH₂ | SEQ ID NO:11 |

### Example (i)

Using the approach shown in FIG. 1 PNA#2 was coupled to wells of a microtitre tray. On to 2 μg of DNA from the prostate cancer cell lines LNCaP, PC-3-M and DU145 was treated with bisulfite as described (Clark S J, Harrison J, Paul C L and Frommer M. High sensitivity mapping of methylated cytosines. Nucleic Acids Res. 22: 2990-2997 (1994)) and resuspended in 100 μL. DNAs were diluted 1:100 with ExpressHyb buffer and 100 μL samples added to wells for hybridisation. One μg of salmon sperm DNA was used in control wells. After hybridisation for 2 hr and washing steps hybridisation was carried out with PNA#3 coupled to either 0.5 μM or 0.1 μM fluorospheres.

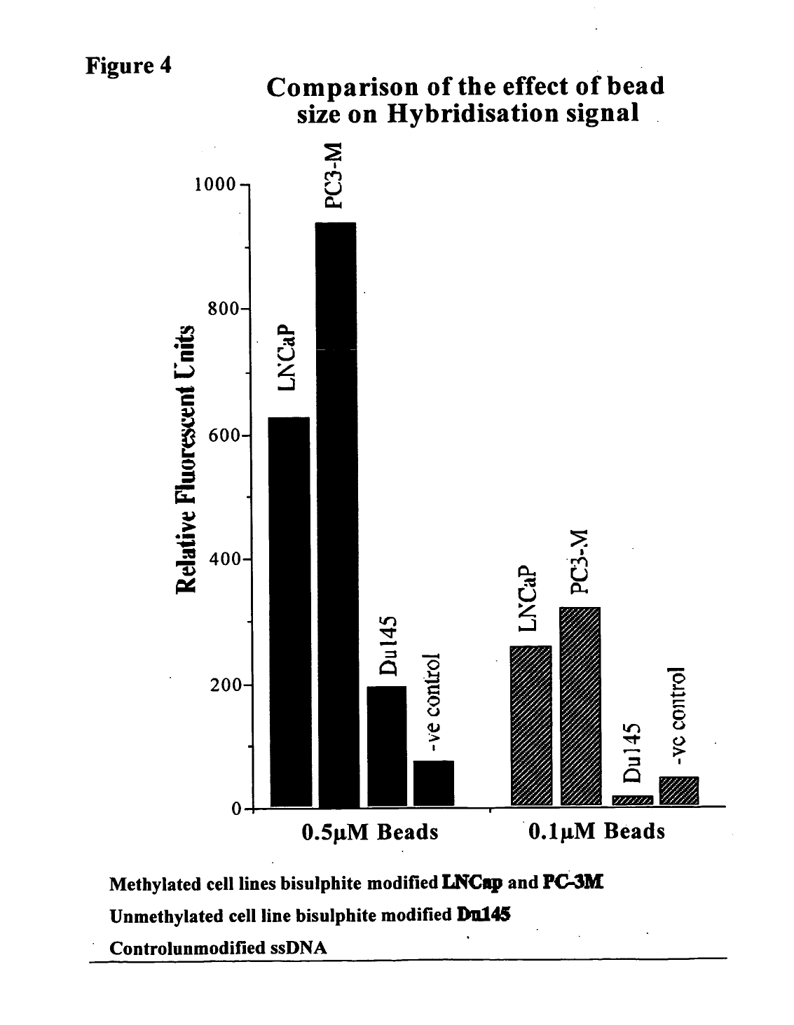

Fluorescent signals were for both LNCaP and PC-3-M DNAs in comparison to DU145 and the negative control salmon sperm DNAs (FIG. 4) after background subtraction. In all cases, a higher signal was seen when the PNA#3 was coupled to the larger diameter (0.5 μM) spheres.

Genomic sequencing has shown that the GSTP1 gene is heavily methylated in LNCaP DNA and significantly methylated in PC-3-M DNA. DNA from the DU145 cell line was shown however to be under methylated (<10%) across the region targeted by the PNA probes used (Lee W H, Morton R A, Epstein J I, Brooks J D, Campbell P A, Bova G S, Hsieh W-S, Isaacs W B and Nelson W G. Cytidine methylation of regulatory sequences near the pi-class glutathione S-transferase gene accompanies prostatic carcinogenesis. PNAS 91:11733-11737 (1994)). The assay is thus able to distinguish between methylated and unmethylated DNAs following bisulfite treatment.

### Example (ii)

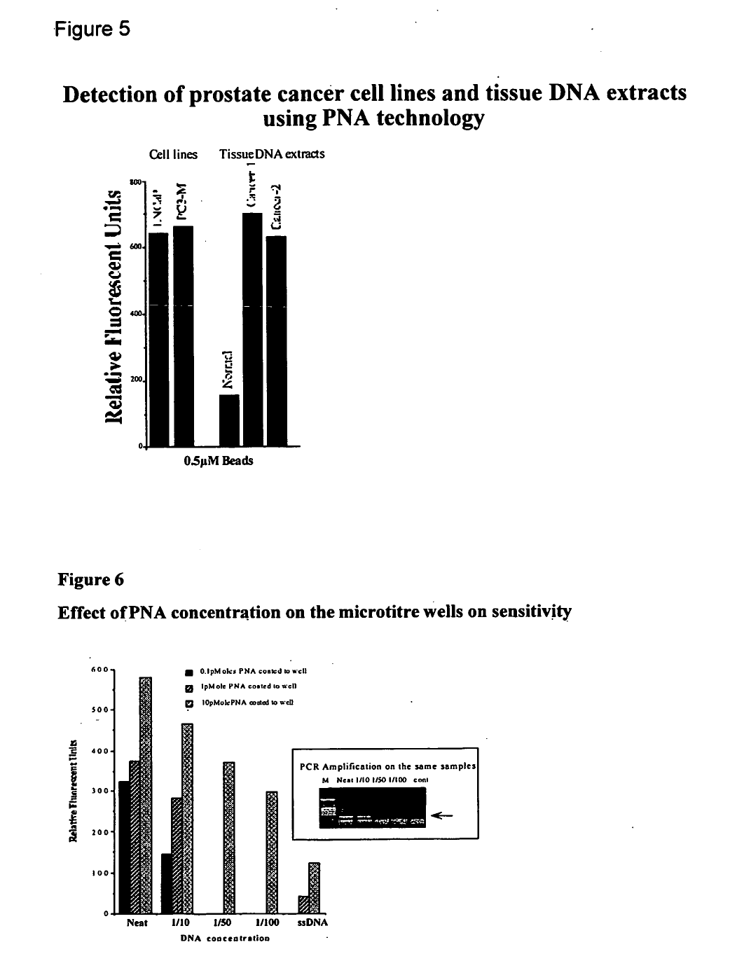

Following the protocol used in Example (i) bisulfite-treated DNA from the LNCaP and PC-3-M cell lines as well as DNA extracted from normal prostate tissue and two samples of prostate cancer tissue were assayed. The normal DNA had been shown to be unmethylated and the cancer DNA samples methylated in the target region. The data (FIG. 5) demonstrate that DNA from normal and cancerous prostate tissue can b distinguished.

### Example (iii)

In FIG. 6 the effects of the level of PNA coated on the wells and of the concentration of the target DNA population were shown. Wells were coated with 0.1, 1 or 10 nmoles of PNA (10 nmoles in previous experiments) and serial dilutions of bisulfite-treated LNCaP DNA. The amounts correspond to 10 ng, 1 ng, 200 pg and 100 pg of DNA prior to bisulfite treatment.

Sensitivity of detection was greatest with 10 pmoles of PNA attached to the wells, with LNCaP DNA corresponding to an input of 100 pg being detectably above the salmon sperm control. Background signals from control salmon sperm DNA also increased as a function of the amount of PNA on the well. PCR amplifications using methylation specific PCR primers were also carried out on the same bisulfite-treated LNCaP DNA samples. The primers used selectively amplify bisulfite-treated DNA corresponding to the GSTP1 promoter methylated at target CpG sites. Primers used were CGPS-5 and CGPS-8 for first round amplification and CGPS-11 and CGPS-12 for second round amplification under conditions as described in PCT/AU99/00306. The data showed that detection by the PNA/fluorescent bead capture assay was at least as sensitive as detection by methylation specific PCR.

II. Detection of Captured DNA Using a Fluorescent Dye

Methylated GSTP1 promoter sequences were detected using bisulfite-treated LNCaP DNA and the single-stranded DNA-binding dye Oligreen (Molecular Probes catalogue number 07582). The Oligreen will bind to any hybridised (captured) DNA remaining after washing steps but will not bind to the PNA probes attached to the wells.

PNA #2 and #3 were coupled to wells of a microtitre plate (1 pMole per well) and 1 μg of bisulfite-treated LNCaP DNA hybridised as above; 1 μg of salmon sperm DNA was used as the control. Hybridisation was done using either ExpressHyb Buffer (Clont ch) or GDA hybridisation buffer (0.75 M NaCl, 0.17 M sodium phosphate, 0.1% (w/v) sodium pyrophosphate, 0.15 M Tris, pH 7.5, 2% sodium dodecyl sulphate, 100 μg/mL salmon sperm DNA, 5×Denhardt's solution [0.1% ficoll, 0.1% bovine serum albumin, 0.1% polyvinylpyrrolidone]). After three washes in 150 μL of water, captured DNA was incubated with a solution of the dye Oligreen that only fluoresces when bound to single stranded DNA. Oligreen stock solution as supplied by the manufacturer was diluted 1:20 in phosphate buffered saline containing 1 mM EDTA and 100 μL added per well. After 5 min incubation fluorescence was read using a 500 nm excitation filter and a 520 nm emission filter.

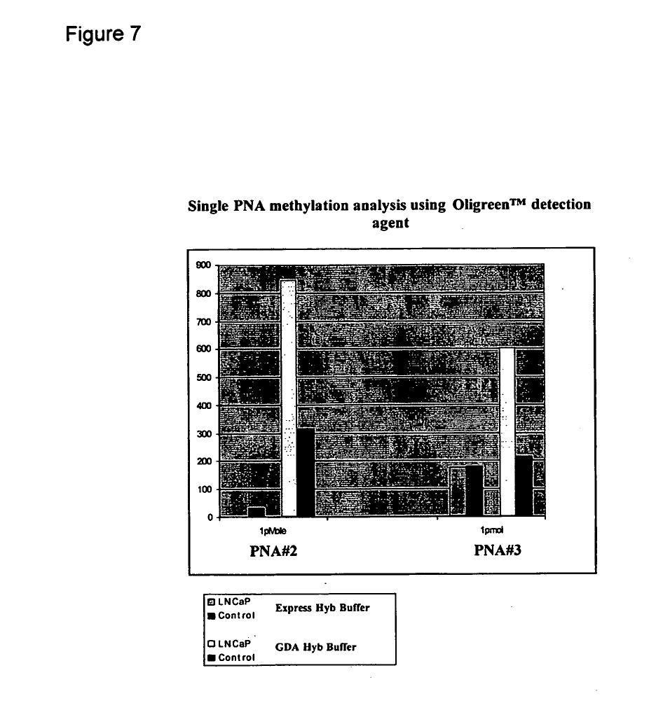

For both PNA#2 and PNA#3 optimal results were obtained using the GDA hybridisation buffer (FIG. 7). In both cases, fluorescence from methylated LNCaP DNA could be detected above the level of control salmon sperm DNA.

III. Detection of Methylated DNA Using Microspheres

Methodology

Referring to FIG. 1 and FIG. 2 approaches for detection of methylated DNA using microspheres is shown.

Coating Microtitre Wells with Capture PNA

(i) The capture PNA#2 (0.0-100 pM per well) in 50 mM Phosphate buffer, 1 mM EDTA pH 8.5 (100 μL) was used to coat N-oxysuccinimide-coated microtitre wells (Costar Cat#2498) for 16-24 hours @ 4° C.

(ii) Plates were washed with 100 μL of 50 mM Phosphate buffer, 1 mM EDTA pH 8.5.

(iii) 150 μL of 3% BSA, 50 mM Phosphate buffer, 1 mM EDTA pH 8.5 was added to each well and the plates left @ 4° C. until required.

Coating the Fluorospheres with Detection PNA

(i) Fluorospheres (Mol cular Probes) were sonicated five times for 5 seconds to break up any aggregated material.

(ii) The detection prob PNA#3 was diluted in a range from 300 pM to 0.3 pM in 250 μL of sonicated 50 mM 2[N-morpholino] thanesulphonic acid (MES) pH 6.0 and 250 μL of sonicated fluorospheres added and the solution left at room temperature for 30 minutes.

(iii) 0.5 mg of 1-ethyl-3[3 dimethylamine propyl] carbodiimide [EDAC], Sigma Cat #E1769, was added to the sample and the sample left 4-6 hours at room temperature in the dark then incubated 16 hours at 4° C.

(iv) 55 μL of 1M glycine was added to the beads and the beads left at room temperature for 2 hours.

(v) The beads were centrifuged for 5-20 minutes (dependant on size of beads, generally 0.5 μM beads required 5 mins while 0.1 μM beads required 20 minutes) at 14,000 rpm in a bench top centrifuge and the supernatant discarded.

(vi) Beads were washed twice with 500 μL of PBS/1% BSA with centrifugation as before between wash steps.

(vii) The beads were then resuspended in 200 μL of PBS/1% BSA and stored at 4° C. in the dark until required.

(viii) Variation of the number of PNAs bound to the beads can be used to optimise sensitivity and minimise background levels.

Hybridisation of DNA

(i) Either control salmon sperm DNA or DNA that was bisulfite treated as in Clark et al (Clark S J, Harrison J, Paul C L and Frommer M. High sensitivity mapping of methylated cytosines. Nucleic Acids Res. 22: 2990-2997 (1994)) was hybridised with PNAs coupled to microtitre wells then added to p r well.

(i) DNA samples were mixed with 100 μL of ExpressHyb™ buffer (Clontech), added to the wells and the plate cov red with cling film or the wells overlayed with mineral oil (Sigma) for long r incubations and the samples incubated at between 45-60° C. for between 1-16 hours.

(iii) Wells were then washed twice with 150 μL of 2×SSC/0.1% SDS @ 45-60° C. for 5-10 minutes per wash.

(iv) The wells were further washed with 150 μL of 0.1×SSC/0.1% SDS @ 45-60° C. for 5-10 minutes and the wash solution discarded.

(i) The PNA/fluorospheres were diluted 1/100 in ExpressHyb™ buffer (Clontech) and 100 μL of samples added to the wells. The plates were covered with cling film or the wells overlayed with mineral oil (Sigma) for longer incubations and the samples incubated @ between 45-60° C. for between 1-16 hours.

(vi) Wells were then washed twice with 150 μL of 2×SSC/0.1% SDS at 45-60° C. for 5-10 minutes per wash.

(vii) The wells were further washed with 150 μL of 0.1×SSC/0.1% SDS at 45-60° C. for 5-10 minutes and the wash solution discarded.

(viii) Finally the fluorescent intensity of each well was measured at the appropriate excitation/emission wave-length for the particular bead (500/520 for yellow beads) in a Victor II fluorescent plate reader.

(ix) Background values measured in wells to which no PNA had been attached were subtracted from all readings.

IV. Method for the Production of In-House Coated Radiolabelled Beads

(i) A specific oligonucleotide (or PNA) is synthesised against the target DNA region of interest. This oligonucleotide contains a 3′ amine group synthesised using standard chemistry (Sigma Genosys).

(ii) The oligonucleotide (or PNA) is then 5′ kinased using gamma P³²dATP as follows:

| | |
|---|---|
| Oligonucleotide (20 ng/μL) | 1 μL |
| ×10 PNK buffer | 1 μL |
| T4 PNK | 1 μL |
| Gamma P³²dATP | 2 μL |
| Sterile water | 5 μL |

(iii) The sample is then incubated at 37° C. for 1 hour then heated to 95° C. for 5 minutes to inactivate the enzyme.

(iv) 0.1 μM carboxylate modified fluorescent beads (Molecular Probes Cat# F-8803) are diluted 1/10,000, 1/100,000 and 1/1,000,000 in sterile water then the kinased oligonucleotide coupled to the beads as follows:

| | |
|---|---|
| Beads | 1 μL |
| Labelled oligo | 3 μL |
| 50 mM MES pH 8.0 | 5 μL |
| 10 mg/mL EDC (Pierce) | 2 μL |

(v) The beads are then incubated @ room temperature for 1 hour to allow the kinased oligonucleotide to attach to the beads via the 3′ amine.

(vi) The beads are then spun in a microfuge at full speed for 15 minutes to sediment the coated beads.

(vii) The supernatant is removed and the beads washed with 100 μL of PBS solution and spun as above.

(viii) The supernatant is removed and the beads resuspended in 50 μL of PBS.

(ix) The CPM of the coated beads is then measured in a standard scintillation counter using the Cerenkov counting protocol. The beads with the highest activity are then used as a detector system in the assay

The idea behind this protocol is to produce th small st number of beads with the highest specific activity, so that only a few beads are needed to bind to the target sequence in order to generate a detectable signal.

V. Urea and Methyoxyamine Conditions of Use

(i) Typically 2 μg of g nomic DNA is r striction digested with an appropriate enzyme (as determined by the target DNA sequence) under the manufacturers conditions for at least 2 hours in a final volume of 20 μL.

(ii) 2.2 μL of freshly prepared 3 M NaOH (6 g in 50 mL H₂O) is added to the DNA and the sample incubated @ 37° C. for 15 minutes.

(iii) 6.24 g of urea is added to 10 mL of sterile distilled water and the solution mixed until gently until the urea has dissolved.

(iv) 7.6 g of sodium metabisulphite (BDH Analar™) is then added and again the solution mixed gently until the bisulphite had dissolved.

(v) The pH of the reagent is then adjusted to 5.0 with 10 M NaOH and the volume of the reagent made to 20 mL with sterile water.

(vi) 208 μL of the reagent is then added to the digested denatured genomic DNA sample.

(vii) 12 μL of 10-100 mM quinol is added the solution mixed and overlayed with mineral oil and incubated for 16 hours at 55° C. in the dark.

(viii) The mineral oil is then removed and the DNA purified using the Promega Wizard™ DNA purification system according to the manufacturers instructions.

(ix) The DNA is eluted from the column with 50 μL of sterile water then 5.5 μL of 3 M NaOH added and the sample incubated at 37° C. for 15 minutes.

(x) At this stage 1/10 volume of methoxyamine (Sigma) from 1-100 mM can be added and incubated with the NaOH as an agent to minimise the nicking of the bisulphite treated DNA.

(xi) In addition tRNA can or glycog n may be added at this stag to h Ip precipitate the DNA

(xii) The DNA is then precipitated by the addition of 33.5 μL of 5 M NH₄OAcetate pH 7.0 and 330 μL of 100% ethanol.

(xiii) The samples are incubated at −20° C. for at least 1 hour then spun down in a microcentrifuge at full speed for 15 minutes

(xiv) The pellet is then air dried for 5-10 minutes and the DNA resuspended in 10 mM Tris/0.1 mM EDTA pH 8.0 in volumes ranging from 10-100 μL dependent on the downstream processing of the modified DNA.

Variations on the above protocol are set out below.

If very small quantities of DNA or micro-dissected cells are to be bisulfite treated this can be done in a number of ways.

Restriction digestion can be omitted.

Urea can be omitted

Glycogen or tRNA or a combination of both can be added at steps (iv), (viii) and (x).

The bisulfite reaction can be done by encapsulating the DNA to be modified in agarose bead, and the entire reaction carried out while the DNA is in the bead.

The time of the reaction with the bisulphite can be reduced from 16 hours to as little as 1 hour but more usually 4 hours.

### SUMMARY

The methods of the present invention can be applied for the detection of any DNA using one ligand (preferably an oligonucleotide or PNA) bound to a solid support and one coupled to a microsphere. Natural oligonucleotides or PNAs may be used, but PNAs were preferred because of their specificity and rate of hybridisation.

In one particular adaptation, the methods of the invention can be used to distinguish the presence of methylated cytosines in DNA that has been treated with sodium bisulfite. The specificity of hybridisation can be used to discriminate against molecules that have not reacted completely with bisulfit (one or more cytosines not converted to uracil) as well as distinguishing between methylated cytosines at CpG sites (which remain as cytosines) and unmethylat d CpG sit s where the cytosine is converted to uracil.

In another adaptation the methods of the invention can be used to discriminate against DNA whose cytosines have not reacted completely with bisulfite reagent to convert them to uracils because they happen to carry a methyl group in the 5′ position.

As treatment with bisulfite changes the sequence of the DNA by converting all cytosines (but not 5-methyl cytosines) to uracils, specific PNA's can be made which recognise a region having 5 methyl cytosines but which will not recognise the same sequence which happens to have no 5-methyl cytosines.

The methods of the invention can also be applied to the discrimination of mutant alleles of a gene where the sequence of one or both of the oligonucleotides or PNAs will match perfectly with one allele but mismatch with the other.

FIG. 7 demonstrates the high sensitivity of the method of the invention showing sensitivity similar to that achieved using PCR techniques.

The method of the invention has numerous applications as previously described including particular use in devising multiple array chips for rapid detection of the methylation status of bulk DNA samples.

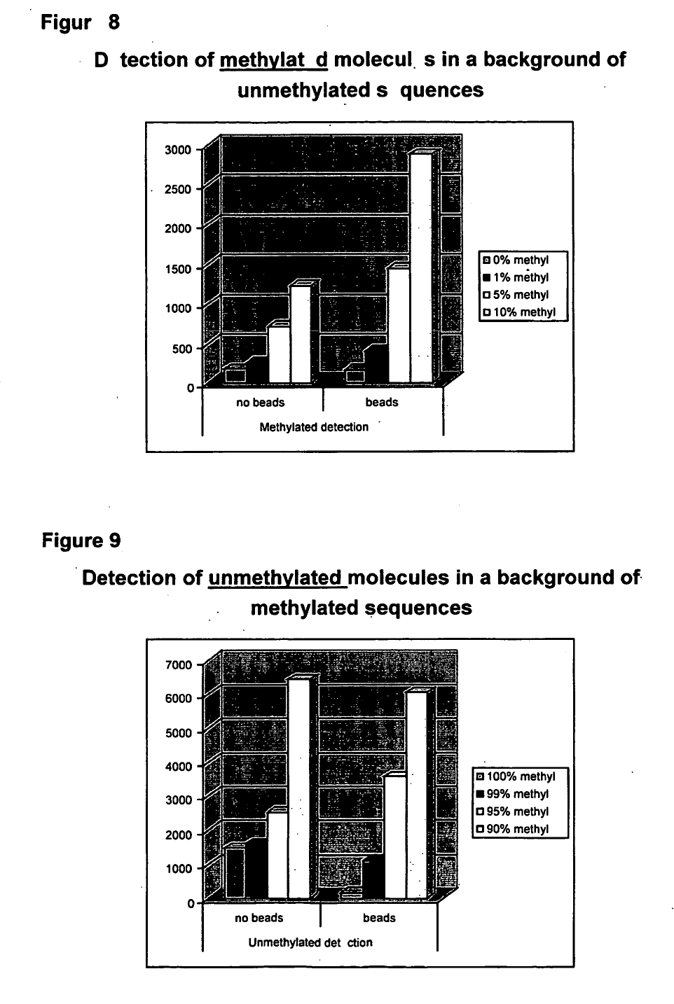

FIG. 8 and FIG. 9 show radioactive data of methylated molecules and unmethylated molecules indicating the sensitivity and specificity of the present invention. As can be seen from the results, the method is capable of distinguishing 1% methylation or unmethylation in a background of 99% unmethylated and 99% methylated molecules, respectively.

Although prostate cancer-related gene was used as an example of the use of the present invention, it will be appreciated that the methods are applicable for many other states and conditions where different methylation states have been found to play a role in disease or altered state of cells. Examples of just some genes affected by CpG island promoter methylation are shown in Table 3. The present invention is clearly applicable for the detection or measurement of such methylation states and many others.

TABLE 3
Examples of genes affected by CpG or CNG
island promoter methylation

| Gene | Location | Cancer | Aging | Comments |
|---|---|---|---|---|
| APC | 5q21 | Colon, gastric, | No | |
| | | oesophageal | | |
| BRACI | 17q21 | Breast, ovarian | No | |
| Calcitonin | 11p15 | Colon, lung, | No | One of the first to be |
| | | haematological | | found methylated in |
| | | | | cancer |
| E-cadherin | 16q22.1 | Breast, gastric, | No | |
| | | thyroid, SCC, | | |
| | | leukemia, liver | | |
| Estrogen | 6q25.1 | Colon, liver, | Yes | Good correlation |
| Receptor | | heart, breast, | | between methylation |
| | | lung | | and loss of expression |
| H19 | 11p15.5 | Wilms tumour | No | Imprinted gene |
| HIC1 | 19p13.3 | Prostate, | Yes | Candidate tumour |
| | | breast, brain, | | suppressor |
| | | lung | | |
| IGF2 | 11p15.5 | Colon, AML | Yes | Has large CpG island |
| MDGI | 1p33-35 | Breast | No | |
| MGMT | 10q26 | Brain, colon, | No | |
| | | lung, breast | | |
| MYOD1 | 11p15.4 | Colon, breast, | Yes | |
| | | bladder, lung | | |
| N33 | 8p22 | Colon, | Yes | Oligo-saccharyl- |
| | | prostate, brain | | transferase |
| P15 | 9q21 | Leukemia, | No | |
| | | lung, colon | | |
| P16 | 9q21 | Lung, colon, | No | Methylation occurs as |
| | | lymphoma, | | frequent as deletions |
| | | bladder, and | | or other mutations |
| | | more | | |
| TIMP3 | 22q12.1 | Brain, kidney | No | |
| WT1 | 11p13 | Breast, colon, | No | |
| | | Wilms tumour | | |

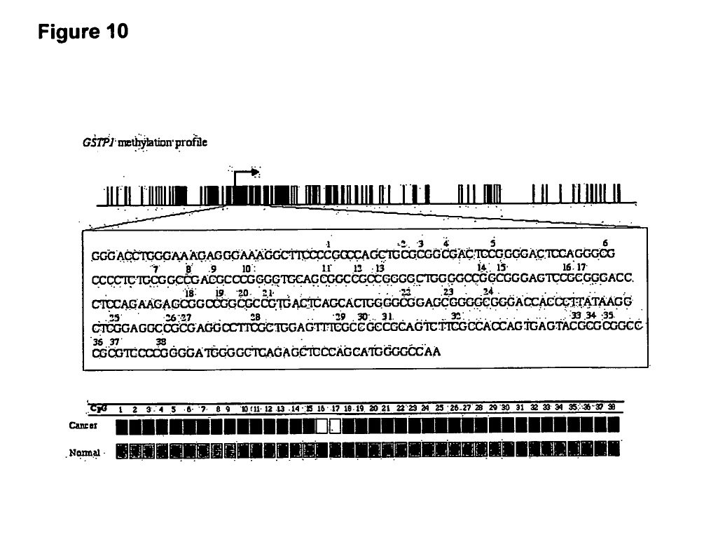

FIG. 10 shows an example of the methylation pattern of GSTP1 in prostate cancer. As can be seen, only subtle changes in the methylation state of this gene region have been implicated in this cancer state. The ability to detect such changes by the methods according to the present invention is a powerful tool for the early detection of cancer and other altered states in cells as well as determining the affect of therapeutic and other agents on cells and tissue.

It will be appreciated by persons skilled in the art that numerous variations and/or modifications may be made to the invention as shown in the specific embodiments without departing from the spirit or scope of the invention as broadly described. The present embodiments were, therefore, to be considered in all respects as illustrative and not restrictive.

1. A method for det cting presence of a target DNA in a sample, the method comprising:
(a) treating a sample containing DNA with an agent that modifies unmethylated cytosine;
(b) providing to the treated sample a detector ligand capable of binding to a target region of DNA and allowing sufficient time for a detector ligand to bind to a target DNA; and
(c) measuring binding of the detector ligand to DNA in the sample to determine the presence of the target DNA in a sample.

2. The method according to claim 1 wherein the sample is selected from the group consisting of biological, tissue, environmental, and microbiological.

3. The method according to claim 2 wherein the biological sample is selected from the group consisting of blood, urine, faeces, semen, cerebrospinal fluid, and cells.

4. The method according to claim 2 wherein the tissue sample is selected from the group consisting of brain, colon, urogenital, lung, renal, hematopoietic, breast, thymus, testis, ovary, and uterus.

5. The method according to claim 4 wherein the tissue sample is selected from the group consisting of brain, colorectal, and prostate.

6. The method according to claim 5 wherein the tissue sample is prostate.

7. The method according to claim 2 wherein the microbiological sample is selected from the group consisting of virus, viroid, bacteria, yeast, fungi, and protozoa.

8. The method according to any one of claims 1 to 7 wherein the modifying agent is capable of modifying unmethylated cytosine but not methylated cytosine.

9. The method according to claim 8 wherein the modifying agent is selected from the group consisting of bisulfite, acetate and citrate.

10. The method according to claim 9 wherein the modifying agent is sodium bisulfite and cytosine is modified to uracil.

11. The method according to claim 10 further including additives selected from the group consisting of urea, methoxyamine and mixtures thereof.

12. The method according to any one of claims 1 to 11 wherein the detector ligand is selected from the group consisting of peptide nucleic acid (PNA), oligonucleotide, modified oligonucleotide, single stranded DNA, RNA, aptamer, antibody, protein, peptide, a combination thereof, and chimeric versions thereof.

13. The method according to claim 12 wherein the detector ligand is a PNA molecule or an oligonucleotide molecule.

14. The method according to claim 13 wherein the detector ligand is a PNA molecule.

15. The method according to claim 14 wherein the PNA is from 5 to 40 bases in length.

16. The method according to claim 14 or 15 wherein the PNA is directed to a CpG- or CNG-containing r gion of DNA.

17. The method according to claim 16 wherein the CpG- or CNG-containing region of DNA is in a regulatory region of a gene or an enhancer of any regulatory element or region selected from the group consisting of promoter, enhancer, oncogene, or other regulatory element which activity is altered by environmental factors including chemicals, toxins, drugs, radiation, synthetic or natural compounds, and microorganisms or other infectious agents including viruses, bacteria, yeast, fungi, protozoa, and prions.

18. The method according to any one of claims 1 to 17 wherein the detector ligand contains a detectable label and the binding of the ligand to target DNA is detected by measuring the presence and/or amount of the detectable label associated with the DNA.

19. The method according to claim 18 wherein the detectable label is selected from the group consisting of fluorescence, radioactivity, enzyme, hapten and dendrimer.

20. A method for estimating extent of methylation of a target DNA in a sample, the method comprising:
(a) treating a sample containing DNA with an agent that modifies unmethylated cytosine;
(b) providing to the treated sample a detector ligand capable of distinguishing between methylated and unmethylated cytosine of DNA and allowing sufficient time for a detector ligand to bind to a target DNA; and
(c) detecting binding of the detector ligand to DNA in the sample such that the degree or amount of binding is indicative of the extent of methylation of the target DNA.

21. The method according to claim 20 wherein the sample is selected from the group consisting of biological, tissue, environmental, and microbiological.

22. The method according to claim 21 wherein the biological sample is selected from the group consisting of blood, urin , faeces, semen, cerebrospinal fluid, and cells.

23. The method according to claim 22 wherein the tissue sample is selected from the group consisting of brain, colon, urogenital, lung, renal, hematopoietic, breast, thymus, testis, ovary, and uterus.

24. The method according to claim 23 wherein the tissue sample is selected from the group consisting of brain, colorectal, and prostate.

25. The method according to claim 24 wherein the tissue sample is prostate.

26. The method according to claim 21 wherein the microbiological sample is selected from the group consisting of virus, viroid, bacteria, yeast, fungi., and protozoa.

27. The method according to any one of claims 20 to 26 wherein the modifying agent is capable of modifying unmethylated cytosine but not methylated cytosine.

28. The method according to claim 27 wherein the modifying agent is selected from the group consisting of bisulfite, acetate and citrate.

29. The method according to claim 28 wherein the modifying agent is sodium bisulfite and cytosine is modified to uracil.

30. The method according to claim 29 further including additives selected from the group consisting of urea, methoxyamine and mixtures ther of.

31. The method according to any one of claims 20 to 30 wherein the detector ligand is selected from the group consisting of peptide nucleic acid (PNA), oligonucleotid , modified oligonucleotid , single stranded DNA, RNA, aptamer, antibody, protein, peptide, a combination thereof, and chimeric versions thereof.

32. The method according to claim 31 wherein the detector ligand is a PNA molecule or an oligonucleotide molecule.

33. The method according to claim 32 wherein the detector ligand is a PNA molecule.

34. The method according to claim 33 wherein the PNA is from 5 to 40 bases in length.

35. The method according to claim 33 or 34 wherein the PNA is directed to a CpG- or CNG-containing region of DNA.

36. The method according to claim 35 wherein the CpG- or CNG-containing region of DNA is in a regulatory region of a gene or an enhancer of any regulatory element or region selected from the group consisting of promoter, enhancer, oncogene, or other regulatory element which activity is altered by environmental factors including chemicals, toxins, drugs, radiation, synthetic or natural compounds, and microorganisms or other infectious agents including viruses, bacteria, yeast, fungi, protozoa, and prions.

37. The method according to any one of claims 20 to 36 wherein the detector ligand contains a detectable label and the binding of the ligand to target DNA is det cted by measuring th presence and/or amount of the detectable label associated with the DNA.

38. The method according to claim 37 wherein the detectable label is selected from the group consisting of fluorescence, radioactivity, enzyme, hapten and dendrimer.

39. The method according to any one of claims 20 to 38 wherein two different detector ligands are used, wherein a first ligand being capable of binding to a region of DNA that contains one or more methylated cytosines and a second ligand being capable of binding to a corresponding region of DNA that contains no methylated cytosines.

40. The method according to claim 39 wherein the two ligands are added to the same treated sample and the binding of each ligand is detected in the one treated sample.

41. The method according to claim 39 wherein each ligand is added to a separate assay and the binding of each ligand is detected in each assay and the binding of the two ligands is compared.

42. A method for detecting the presence of a target DNA in a sample, the method comprising:
(a) treating a sample containing DNA with an agent that modifies unmethylated cytosine;
(b) providing a support to which is bound a capture ligand capable of recognising a first part of a target DNA sequence;
(c) contacting the support with the treated sample for sufficient time to allow DNA to bind to a capture ligand such that target DNA in the sample binds to the support via the capture ligand;
(d) contacting the support with a detector ligand capable of recognising a second part of the target DNA sequence and allowing sufficient time for a detector ligand to bind to a target DNA; and
(e) measuring binding of the detector ligand to DNA bound to the support to determine the presence of the target DNA in the sample.

43. The method according to claim 42 wherein the sample is selected from the group consisting of biological, tissue, environmental, and microbiological.

44. The method according to claim 43 wherein the biological sample is selected from the group consisting of blood, urine, faeces, semen, cerebrospinal fluid, and cells.

45. The method according to claim 43 wherein the tissue sample is selected from the group consisting of brain, colon, urogenital, lung, renal, hematopoietic, breast, thymus, testis, ovary, and uterus.

46. The method according to claim 45 wherein the tissue sample is selected from the group consisting of brain, colorectal and prostate.

47. The method according to claim 46 wherein the tissue sample is prostate.

48. The method according to claim 43 wherein the microbiological sample is selected from the group consisting of virus, viroid, bacteria, yeast, fungi, and protozoa.

49. The method according to any one of claims 42 to 48 wherein the modifying agent is capable of modifying unmethylated cytosine but not methylated cytosine.

50. The method according to claim 49 wherein the modifying agent is selected from the group consisting of bisulfite, acetate and citrate.

51. Th method according to claim 50 wherein the modifying agent is sodium bisulfite and cytosine is modified to uracil.

52. The method according to claim 51 further including additives selected from the group consisting of urea, methoxyamine, and mixtures thereof.

53. The method according to any one of claims 42 to 52 wherein the support is selected from the group consisting of glass, polymer including cellulose, polyacrylamide, nylon, polystyrene, polyvinyl chloride or polypropylene, plastic materials, fluorescent beads, magnetic beads, synthetic or natural membranes, latex beads, column supports, beads or slides, nanotubes, fibres, and organic or inorganic solid supports.

54. The method according to claim 53 wherein the support is a magnetic bead or a fluorescent bead.

55. The method according to any one claims 42 to 53 wherein step (b) comprises a plurality capture ligands arrayed on a solid support.

56. The method according to claim 55 wherein the array contains multiple copies of the same ligand so as to capture the same target DNA on the array.

57. The method according to claim 55 wherein the array contains a plurality of different ligands targeted to different DNA so as to capture a plurality of target DNA molecules on the array.

58. The method according to any one of claims 55 to 57 wherein the array contains from 10 to 1 0000 capture ligands.

59. The method according to claim 58 wher in the array has less than 500 capture ligands.

60. The method according to any one of claims 42 to 59 wherein the capture ligand is selected from the group consisting of peptide nucleic acid (PNA), oligonucleotide, modified oligonucleotide, single stranded DNA, RNA, aptamer, antibody, protein, peptide, a combination thereof, and chimeric versions thereof.

61. The method according to claim 60 wherein the capture ligand is a PNA molecule or an oligonucleotide molecule.

62. The method according to claim 61 wherein the capture ligand is a PNA molecule.

63. The method according to claim 62 wherein the PNA is from 5 to 40 bases in length.

64. The method according to any one of claims 42 to 63 wherein the detector ligand is selected from the group consisting of peptide nucleic acid (PNA), oligonucleotide, modified oligonucleotide, single stranded DNA, RNA, aptamer, antibody, protein, peptide, a combination thereof, and chimeric versions thereof.

65. The method according to claim 64 wherein the detector ligand is a PNA molecule or an oligonucleotide molecule.

66. The method according to claim 65 wherein the detector ligand is a PNA molecule.

67. The method according to claim 66 wherein the PNA is from 5 to 40 bases in length.

68. The method according to claim 66 or 67 wherein the PNA is directed to a CpG- or CNG-containing region of DNA.

69. The method according to claim 68 wherein the CpG- or CNG-containing region of DNA is in a regulatory region of a gene or an enhancer of any regulatory element or region selected from the group consisting of promoter, enhancer, oncogene, or other regulatory element which activity is altered by environmental factors including chemicals, toxins, drugs, radiation, synthetic or natural compounds, and microorganisms or other infectious agents including viruses, bacteria, yeast, fungi, protozoa, and prions.

70. The method according to any one of claims 42 to 69 wherein the detector ligand contains a detectable label and the binding of the ligand to target DNA is detected by measuring the presence and/or amount of the detectable label.

71. The method according to claim 70 wherein the detectable label is selected from the group consisting of fluorescence, radioactivity, enzyme, hapten and dendrimer.

72. The method according to any one of claims 42 to 71 wherein two different detector ligands are used, wherein a first ligand being capable of binding to a region of DNA that contains one or more methylated cytosines and a second ligand being capable of binding to a corresponding region of DNA that contains no methylated cytosines.

73. The method according to claim 72 wherein the two detector ligands are added to the same treated sampl and the binding of each ligand is detected in the one treated sample.

74. The method according to claim 72 wh rein each detector ligand is added to a separate assay and the binding of each ligand is detected in each assay and the binding of the two ligands is compared.

75. A method for estimating extent of methylation of a target DNA in a sample, the method comprising:
(a) treating a sample containing DNA with an agent that modifies unmethylated cytosine;
(b) providing a support to which is bound a capture ligand which is capable of recognising a first part of a target DNA sequence;
(c) contacting the support with the treated sample for sufficient time to allow DNA to bind to a capture ligand such that target DNA in the sample binds to the support via the capture ligand;
(d) contacting the support with a detector ligand capable of distinguishing between methylated and unmethylated cytosine of DNA such that the detector ligand binds to any target DNA on the support; and
(e) detecting binding of the detector ligand to the support such that the degree or amount of binding is indicative of the extent of methylation of the target DNA.

76. The method according to claim 75 wherein the sample is selected from the group consisting of biological, tissue, environmental, and microbiological.

77. The method according to claim 76 wherein the biological sample is selected from the group consisting of blood, urine, faeces, semen, cerebrospinal fluid, and cells.

78. The method according to claim 77 wherein the tissue sample is selected from the group consisting of brain, colon, urogenital, lung, renal, hematopoietic, breast, thymus, testis, ovary, and uterus.

79. The method according to claim 78 wherein the tissue sample is selected from the group consisting of brain, colorectal, and prostate.

80. The method according to claim 79 wherein the tissue sample is prostate.

81. The method according to claim 77 wherein the microbiological sample is selected from the group consisting of virus, viroid, bacteria, yeast, fungi, and protozoa.

82. The method according to any one of claims 75 to 81 wherein the modifying agent is capable of modifying unmethylated cytosine but not methylated cytosine.

83. The method according to claim 82 wherein the modifying agent is selected from the group consisting of bisulfite, acetate and citrate.

84. The method according to claim 83 wherein the modifying agent is sodium bisulfite and cytosine is modified to uracil.

85. The method according to claim 84 further including additives selected from the group consisting of urea, methoxyamine, and mixtures thereof.

86. The method according to any one of claims 75 to 85 wherein the support is selected from the group consisting of glass, polymer including cellulose, polyacrylamide, nylon, polystyrene, polyvinyl chloride or polypropylene, plastic materials, fluorescent beads, magnetic beads, synthetic or natural membranes, latex beads, column supports, beads or slides, nanotub s, fibres, and organic or inorganic solid supports.

87. The method according to claim 86 wherein the support is a magnetic bead or a fluorescent bead.

88. The method according to any one claims 75 to 87 wherein step (b) comprises a plurality capture ligands arrayed on a solid support.

89. The method according to claim 88 wherein the array contains multiple copies of the same ligand so as to capture the same target DNA on the array.

90. The method according to claim 89 wherein the array contains a plurality of different ligands targeted to different DNA so as to capture a plurality of target DNA molecules on the array.

91. The method according to any one of claims 75 to 90 wherein the array contains from 10 to 10,000 capture ligands.

92. The method according to claim 91 wherein the array has less than 500 capture ligands.

93. The method according to any one of claims 15 to 92 wherein the capture ligand is selected from the group consisting of peptide nucleic acid (PNA), oligonucleotide, modified oligonucleotide, single stranded DNA, RNA, aptamer, antibody, protein, peptide, a combination thereof, and chimeric versions thereof.

94. The method according to claim 93 wherein the capture ligand is a PNA molecule or an oligonucleotide molecule.

95. The method according to claim 94 wherein the capture ligand is a PNA molecule.

96. The method according to claim 95 wherein the PNA is from 5 to 40 bases in length.

97. The method according to any one of claims 75 to 96 wherein the detector ligand is selected from the group consisting of peptide nucleic acid (PNA), oligonucleotide, modified oligonucleotide, single stranded DNA, RNA, aptamer, antibody, protein, peptide, a combination thereof, and chimeric versions thereof.

98. The method according to claim 97 wherein the detector ligand is a PNA molecule or an oligonucleotide molecule.

99. The method according to claim 98 wherein the detector ligand is a PNA molecule.

100. The method according to claim 99 wherein the PNA is from 5 to 40 bases in length.

101. The method according to claim 98 or 99 wherein the PNA is directed to a CpG- or CNG-containing region of DNA.

102. The method according to claim 101 wherein the CpG- or CNG-containing region of DNA is in a regulatory region of a gene or an enhancer of any regulatory element or region selected from the group consisting of promoter, enhancer, oncogene, or other regulatory element which activity is altered by environmental factors including chemicals, toxins, drugs, radiation, synthetic or natural compounds, and microorganisms or other infectious agents including viruses, bacteria, yeast, fungi, protozoa, and prions.

103. The method according to any one of claims 75 to 102 wherein the detector ligand contains a detectabl label and the binding of the ligand to targ t DNA is d tected by measuring the presence and/or amount of the detectable label.

104. The method according to claim 103 wherein the detectable label is selected from the group consisting of fluorescence, radioactivity, enzyme, hapten and dendrimer.

105. The method according to any one of claims 75 to 104 wherein two different detector ligands are used, wherein a first ligand being capable of binding to a region of DNA that contains one or more methylated cytosines and a second ligand being capable of binding to a corresponding region of DNA that contains no methylated cytosines.

106. The method according to claim 106 wherein the two detector ligands are added to the same treated sample and the binding of each ligand is detected in the one treated sample.

107. The method according to claim 106 wherein each detector ligand is added to a separate assay and the binding of each ligand is detected in each assay and the binding of the two ligands is compared.

108. A method for detecting a methylated CpG- or CNG-containing DNA, the method comprising:
(a) treating a sample containing DNA with bisulfite to modify unmethylated cytosine to uracil in the DNA;
(b) providing to the treated sample a detector PNA ligand capable of distinguishing between methylated and unmethylated cytosine of DNA; and
(c) detecting the methylated DNA based on the presence or absence of binding of the detector PNA ligand.

109. Th method according to claim 108 wherein the detector PNA ligand is capable of binding to a methylated CpG- or CNG-containing DNA but not to a corresponding unmethylated CpG- or CNG-containing DNA. and binding of the PNA ligand to DNA is indicative of methylation of th DNA.

110. A method for estimating extent of methylation of a target DNA in a sample, the method comprising:
(a) treating a sample containing DNA with bisulfite to modify unmethylated cytosine to uracil;
(b) providing a solid support in the form of a magnetic bead to which is bound a capture PNA or oligonucleotide ligand which is capable of recognising a first part of a target DNA sequence;
(c) contacting the support with the treated sample suspected of containing the target DNA such that target DNA in the sample binds to the support via the capture ligand;
(d) contacting the support with a detector PNA ligand capable of distinguishing between methylated and unmethylated cytosine of DNA; and
(e) determining the extent of methylation of the DNA bound to the support by measuring the amount of bound detector ligand.
